(19) United States
(12) Patent Application Publication
(10) Pub. No.: US 2026/0167730 A1
(43) Pub. Date: Jun. 18, 2026

(54) ALK7 BINDING PROTEINS

(71) Applicant: Novo Nordisk A/S, Bagsvaerd (DK)

(72) Inventors: Hans Dennis Aasberg, Frederikssund (DK); Tileli Amimeur, Seattle, WA (US); Laust Bruun Johnsen, Skodsborg (DK); Anette Henriksen, Alleroed (DK)

(21) Appl. No.: 19/422,546

(22) Filed: Dec. 17, 2025

Related U.S. Application Data
(60) Provisional application No. 63/735,344, filed on Dec. 18, 2024.

(30) Foreign Application Priority Data
Jan. 10, 2025 (EP) 25151217.4

(51) Int. Cl. C07K 16/28; A61K 39/00; A61P 3/04

(52) U.S. Cl. CPC C07K 16/2863; A61P 3/04; A61K 2039/505; C07K 2317/24; C07K 2317/92

(57) ABSTRACT

The present disclosure provides anti-ALK7 antibody or antigen binding fragment thereof. The anti-ALK7 antibodies or antigen binding fragments thereof show a distinct binding pattern to the ECD of ALK7 and favourable functionality. The anti-ALK7 antibodies or antigen binding fragments thereof inhibit ALK7 signalling and lend themselves to pharmaceutical use in treatment and/or prevention of metabolic disorders, such as type 2 diabetes, obesity, and cardiovascular diseases.

### CROSS-REFERENCE TO RELATED APPLICATIONS

This application claims priority under 35 U.S.C. § 119 to U.S. Provisional Application No. 63/735,344, filed Dec. 18, 2024, and claims priority to European Patent Application 25151217.4, filed Jan. 10, 2025; all of which are hereby incorporated by reference.

### TECHNICAL FIELD

The present disclosure provides ALK7 binding proteins, such as anti-ALK7 antibodies or antigen binding fragments thereof, with improved properties and their use in methods of treatment.

### INCORPORATION-BY-REFERENCE OF THE SEQUENCE LISTING

The instant application contains a Sequence Listing which has been submitted in XML format via USPTO patent electronic filing system and is hereby incorporated by reference in its entirety. Said XML file, created on Dec. 15, 2025, is named “240012US02.xml”, and is 98,968 bytes in size.

### BACKGROUND

Activin receptor-like kinase 7 (ALK7, also known ACVR1C) is a single transmembrane type 1 serine/threonine kinase within the TGF-β receptor family, primarily expressed on mature adipocytes in humans and relevant animal models e.g. rodents. The signalling complex is a heterooligomer that consists of a heterotetrameric receptor complex with two type I and two type II transmembrane serine/threonine kinases that is activated by homodimeric ligands of the TGF-β superfamily, including Activin B, Activin C, and Activin E. In the signalling complex, the type II receptors phosphorylate and activate the type I receptors, which phosphorylate and then bind and activate the SMAD transcriptional regulators, SMAD2 and SMAD3.

Activation of ALK7 promote lipid storage in adipose tissue. Inhibition of the ALK7, including inhibition with neutralizing antibodies, has been shown to reduce body weight and have other metabolic benefits in pre-clinical animal models. Human genetic evidence supports ALK7 inhibition as a potential target for metabolic disease. Rare-frequency loss of function/missense mutations are associated with reduced waist-hip ratio (WHR) adjusted for BMI and decreased risk of developing type 2 diabetes, suggesting an improved metabolic profile in these individuals. Inhibition or silencing of ALK7 leads to increased lipolysis from adipocytes and weight loss in the diet-induced obese mouse model. (See e.g. Diabetes, 68 (1), 226, 2018, Nature Communications, 13 (1), 4319, 2022 and JCI Insights, 8 (4), e161229, 2023.)

Some antibodies to ALK7 have previously been described and characterized in patent applications such as WO17/1805037, WO19/084249 and WO20/244343.

### SUMMARY

The present disclosure provides antigen binding proteins, including antibodies binding the extracellular domain of human ALK7. This disclosure, in particular, provides antibodies that are potent ALK7 inhibitors. In particular, the present disclosure provides anti-ALK7 antibodies or antigen binding fragments thereof.

The present disclosure provides an anti-ALK7 antibody or antigen binding fragment thereof comprising three heavy chain CDR sequences, hCDR1-3, and three light chain CDR, sequences, ICDR1-3, wherein
a) hCDR1 is SGSYWN (SEQ ID NO.: 63) with up to 1 or 2 amino acid substitutions,
b) hCDR2 is YISFDGRTNYNPSLKN (SEQ ID NO.: 65) with up to 1, 2 or 3 amino acid substitutions,
c) hCDR3 is DYYGSEGFAY (SEQ ID NO.: 72) with up to 1, 2 or 3 amino acid substitutions,
d) ICDR1 is KSSQSLLVSRTRKNYLA (SEQ ID NO.: 74) with up to 1, 2 or 3 amino acid substitutions,
e) ICDR2 is WTSTRES (SEQ ID NO.: 76) with up to1 or 2 amino acid substitutions, and
f) ICDR3 is KQSYNLPYT (SEQ ID NO.: 78) with up to 1 or 2 amino acid substitutions.

The present disclosure provides an anti-ALK7 antibody or antigen binding fragment thereof that binds a non-contiguous ALK7 epitope, such as an ALK7 epitope comprising
i. at least two amino acid residues selected from the group consisting of L26, C30, L32, C33, S35, S36, C40, Q41, T42, E43, G44, and W47,
ii at least two amin acid residues selected from the group consisting of S62, C63, V64, S65, L66, P67, E68, N70, A71, and F74; and
iii. at least one amino acid residue selected from the group consisting of F88 and T89.

The present disclosure provides an anti-ALK7 antibody or antigen binding fragment thereof, which paratope comprises heavy chain amino acid residues selected from the group consisting of S28, T30, S31, G32, Y34, Y50, F53, D54, R56, N58, Y97, G98, S99, and E100, and light chain amino acid residues selected from the group consisting of N27D, R27F, T28, K30, Y32, W50, S91, Y92, N93, L94, and Y96 with up to 1 or 2 amino acid substitutions according to the Kabat numbering scheme.

The present disclosure provides an ALK7 antibody or antigen binding fragment thereof, having an IC50 below 50 nM, such as below 10 nM when measured in the reporter gene assay (RGA) of Example 4.

The present disclosure provides a pharmaceutical composition comprising an anti-ALK7 antibody or antigen binding fragment thereof as describe herein.

The present disclosure provides ALK7 binding proteins, such as anti-ALK7 antibodies or antigen binding fragments thereof with biophysical properties suitable for subcutaneous administration.

The present disclosure provides medical uses of an ALK7 antibody as describe herein including prevention and treatment of type 2 diabetes, metabolic diseases and cardiovascular diseases (CVD).

### BRIEF DESCRIPTION OF DRAWINGS

FIG. 1A shows the 5 Å Euclidian distance cut-off epitope of the Fab A—ECD of ALK7 crystal complex, highlighting the role of CDR L1 in the epitope-paratope interface.

FIG. 1B shows the bent shape of the epitope: paratope contact surface of the Fab A—ALK7 ECD crystal complex.

FIG. 2 shows the weight loss observed in DIO mice after 28 days of treatment with once weekly dosing of 1, 5 or 10 mg/kg (4 dosages each) of V13 as described in Example 5B.

### DEFINITIONS

The term “antibody”, as referred to herein, comprises whole antibodies and antigen-binding fragments thereof, or single chains thereof (see further below). Full-length antibodies (or whole antibodies) comprise four polypeptide chains, two heavy (H) chains and two light (L) chains interconnected by disulfide bonds. Each heavy chain is comprised of a heavy chain variable region (VH) and a heavy chain constant region (CH). Each light chain is comprised of a light chain variable region (VL) and a light chain constant region (CL). The heavy chain constant region is comprised of three domains, CH1, CH2 and CH3. The variable regions of the heavy and light chains form an antigen binding domain that interacts with the antigen. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). The term “framework region” or “FR” residues refer to those VH or VL amino acid residues that are not within the CDRs, as defined herein.

Each VH and VL is composed of three CDRs and four FRs, arranged from amino-terminus to carboxy-terminus in the following order: FR1, CDR1, FR2, CDR2, FR3, CDR3, FR4.

The carboxy-terminal portion of each of the heavy and light chain defines a constant region primarily responsible for effector function via binding of the immunoglobulin to host tissues or factors, including various cells of the immune system (e.g., effector cells) and the first component (Clq) of the classical complement system. A constant region can include any or all of a CH1 region, hinge region, CH2 region, and CH3 region.

As mentioned above, the term “antibody” is used to describe whole antibodies and antigen-binding fragments thereof, or single chains modification thereof, which specifically binds its corresponding antigen. Examples of antigen-binding fragments include Fab, Fab′, F(ab)₂, F(ab′)₂, F(ab)S, Fv (typically the VL and VH domains of a single arm of an antibody), single-chain Fv (scFv), dsFv, Fd (typically the VH and CHI domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies, camel IgG; IgNAR; as well as one or more isolated CDRs or a functional paratope, where the isolated CDRs or antigen-binding residues or polypeptides can be associated or linked together so as to form a functional antibody fragment.

Various types of antibody fragments have been described or reviewed in, e.g., Holliger and Hudson, Nat Biotechnol 2005;2S:1126-1136; WO2005040219, and published U.S. patent applications 20050238646 and 20020161201.

A full-length antibody has two arms and is thus bivalent, while the antigen binding fragments described above might be polyvalent, bivalent or monovalent.

The term “complementarity-determining region” (“CDR”) or “hypervariable region”, when used herein, refers to the amino acid residues of an antibody that are responsible for antigen binding. The CDRs are considered to be comprised of amino acid residues 24-34 (L1), 50-56 (L2) and 89-97 (L3) in the light-chain variable domain and 31-35B (H1), 50-65 (H2) and 95-102 (H3) in the heavy-chain variable domain; (Kabat numbering, Kabat et al. (1991) Sequences of Proteins of Immunological Interest, Fifth Edition, U.S. Department of Health and Human Services, NIH Publication No. 91-3242) and/or those CDR residues defined from the unique IMGT numbering scheme (residues 26-38 (L1 and H1), 56-55 (L2 and H2) and 105-116 (germline H3 and L3) or 105-117 (rearranged VJ and VDJ genes);) (IMGT numbering, Lefranc, Immunology Today, 18, 509 (1997); Lefranc, The Immunologist, 7, 132-136 (1999); Lefranc, et al., Dev Comp Immunol, 27, 55-77 (2003))

Typically, the numbering of amino acid residues in this region is performed by the method described in Kabat et al., supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The fragment crystallizable region (“Fc region” or “Fc domain”) of an antibody is the “tail” region of an antibody that interacts with cell surface receptors called Fc receptors, as well as some proteins of the complement system.

Monoclonal antibodies are traditionally made by fusing myeloma cells with the spleen cells from a mouse that has been immunized with the desired antigen. Human monoclonal antibodies can be obtained from transgenic animals (e.g. mice or other suitable species) encoding human antibodies. Alternatively, recombinant monoclonal antibodies can be made involving technologies, referred to as repertoire cloning or phage display/yeast display or single B-cell discovery. Recombinant antibody engineering involves the use of viruses or yeast to create antibodies, rather than mice. When initially expressed, this variable region is typically linked to a cleavable signal peptide. The variable region is herein described without the signal peptide.

The terms “humanized antibody”, chimeric antibody” and “human antibody” provides information on the origin of the sequences of the antibody. A humanized antibody”, refers to a human/non-human chimeric antibody that contains sequences, usually at least the minimal complementarity-determining regions (CDR sequences) derived from a non-human germ line immunoglobulin sequence. An antibody is “chimeric antibody” if the antibody light and heavy chain genes have been constructed, typically by genetic engineering, from immunoglobulin variable and constant region genes that originate from different species. For example, the variable segments of genes from a mouse monoclonal antibody may be joined to human constant segments. The term “human antibody”, is intended to describe antibodies having variable regions in which both the framework and CDR regions are derived from human germline immunoglobulin sequences. It is noted that the variable region of such antibodies may none the less comprise amino acid residues which are not found in the human germline sequences due to mutations occurring due to maturation in vivo or in vitro.

Furthermore, if the antibody comprises a constant region, the constant region is usually derived from human germline immunoglobulin sequences. Antibodies provided by the present disclosure may none the less include amino acid residues not encoded by human germline immunoglobulin sequences e.g., mutations introduced by somatic mutation in vivo or by random or site-specific mutagenesis in vitro.

Antibody may thus be produced by various methods including hybridoma cells which includes a B cell obtained from an animal species suitable to generate antibodies, such as mice, rabbits or transgenic nonhuman animals, e.g., a transgenic mouse, having a genome comprising a human heavy chain transgene and a light chain transgene fused to an immortalized cell. Human antibodies may also be isolated from sequence libraries built on selections of human germline sequences, further diversified with natural and synthetic sequence diversity. Human antibodies may further be obtained by in vitro immunisation of human lymphocytes followed by transformation of the lymphocytes with Epstein-Barr virus.

Furthermore, humanized, human and fully human antibodies may comprise residues that are not found in the recipient antibody or in the donor antibody. These modifications are made to further refine antibody performance.

The sequence of any antibody can easily be identified allowing production of the antibody by recombinant methods. Unless otherwise indicated, the recombinant protein, cell culture, and immunological techniques utilized in the present disclosure are standard procedures, well known to those skilled in the art. Post-translational modifications may occur when antibodies are recombinantly expressed in mammalian cells such as Chinese hamster ovary cells, Human Embryonic Kidney cells (HEK) 293, or baby hamster kidney cells. Modifications introduced by recombinant expression can include removal of the C-term Lysine of heavy chains, glycosylation as well as transformation of N-terminal Glutamine (Gln, Q) to pyroglutamic acid which may occur in vitro or in vivo.

The term “antigen” is used broadly herein to describe molecular entities specifically recognized by the antibody, thus including fragments or mimics of the molecule used in the immunization process for raising the antibody as well as molecules used for screenings upon immunization and also molecules used for screening in cases where antibodies are obtained by alternative methods such as phage display screening.

The term “epitope”, as used herein, e.g., an ALK7 epitope, is defined in the context of a molecular interaction between an “antigen binding polypeptide”, such as an antibody, and its corresponding “antigen”. Generally, “epitope” refers to the area or region on an antigen to which an antibody specifically binds, i.e., the area or region in physical contact with the antibody. A protein epitope may comprise amino acid residues in the antigen that are directly involved in binding to the antibody (also called the immunodominant component of the epitope) and other amino acid residues, which are not directly involved in binding, such as amino acid residues of the antigen which are effectively blocked by the Ab (in other words, the amino acid residue is within the “solvent-excluded surface” and/or the “footprint” of the antibody). Definition of protein contacts according to atomic distances is a common strategy in analysis of antibody-antigen interfaces (Gordon et al., Front Immunol 2023; 14:1231623; Akbar et al., Cell Rep 2021, 34, 108856) and recent evidence indicates that a 5 Å cutoff for noncovalent interactions is optimal for building robust protein structure networks (Viloria et al., Sci Rep 2017, 7, 2838). A given antigen may comprise a number of different epitopes, which may include, without limitation; linear peptide antigenic determinants, conformational antigenic determinants which consist of one or more non-contiguous amino acids located near each other in the native (mature) conformation; and post-translational antigenic determinants which consist, either in whole or part, of molecular structures covalently attached to the antigen, such as carbohydrate groups.

The description and definition of epitopes are dependent on the epitope mapping method used, which provide different levels of detail, and it follows that comparison of epitopes for different antibodies on the same antigen can similarly be conducted at different levels of detail.

The terms “binding”, “specifically binding”, “specific binding” and “binding specificity” is used herein to describe the selectivity of an antibody or an antigen binding fragment thereof.

The term “binding affinity” is used as a measure of the strength of a non-covalent interaction between two molecules, e.g. an antibody, or fragment thereof, and an antigen. The term “binding affinity” is used to describe monovalent interactions (intrinsic affinity). Binding affinity between two molecules, e.g. an antibody, or fragments thereof, and an antigen, through a monovalent interaction may be quantified by determination of the dissociation constant (K_{D}). The K_{D} of a monovalent complex can be determined as relationship between the association and dissociation rate constants governing formation of the complex which can be measured by e.g. Surface Plasmon Resonance (SPR). The association rate constant and the dissociation rate constant are referred to as the kₐ (or kₒₙ) and k_{d} (or k_{off}), respectively. K_{D} is related to kₐ and k_{d} through the equation K_{D}=k_{d}/kₐ.

A small or a decrease in the K_{D} value indicates a stronger/higher affinity interaction. A high or an increase in the K_{D} value indicates a weaker/lower affinity interaction.

Typically, the K_{D} for the antibody with respect to the target will be 2-fold, preferably 5-fold, more preferably 10-fold less than K_{D} with respect to another, non-target molecule such as unrelated material or accompanying material in the environment or control. More preferably, the K_{D} will be 50-fold less, such as 100-fold less, or 200-fold less; even more preferably 500-fold less, such as 1,000-fold less, or 10,000-fold less.

Standard assays to evaluate the binding ability of antibodies towards an antigen are known in the art-including, for example, ELISAs, Western blots, RIAs, and flow cytometry analysis. The binding kinetics and binding affinity of the antibody also can be assessed by standard assays known in the art, such as SPR.

A competitive binding assay can be conducted in which the binding of the antibody to the antigen target is compared to the binding of the antigen target by another antibody. The concentration at which 50% inhibition occurs is known as the IC50. Under ideal conditions, the IC50 is equivalent to K_{D}. As the skilled person will appreciate, “avidity” relates to the overall strength of interaction between two molecules, such as an antibody and antigen. Avidity depends on both the affinity and the valency of interactions and is therefore relevant for antibodies with two antigen binding domains.

Further assays for determining functionality of a given antibodies may include cellular based assay which are specific for the given antigen and the effect of antibody binding.

Throughout this specification the word “comprise”, or variations such as “comprises” or “comprising”, will be understood to imply the inclusion of a stated element, integer or step, or group of elements, integers or steps, but not necessarily the exclusion of any other element, integer or step, or group of elements, integers or steps. Unless, not technically sensible, comprise(s)/comprising also imply the stated element, integer or step, or group of elements, integers or steps in isolation and thus also covers embodiment(s) “consist/consisting” of the stated element, integer or step, or group of elements, integers or steps.

In an embodiment, a molecule consists essentially of the defined sequence.

In another embodiment, a molecule consists of the defined sequence.

In an embodiment the molecule such as an antibody or DNA sequence is an isolated molecule. The term “isolated antibody” refers to an antibody that has been separated and/or recovered from another/other component(s) of its natural environment and/or purified from a mixture of components in its natural environment.

Preferred parameters for a protein sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 3,

The Geneious Prime program is useful with the above parameters. The aforementioned parameters are the default parameters for peptide comparisons (along with no penalty for end gaps) using the GAP algorithm.

An “amino acid substitution” involves a substitution of one amino acid residue with another residue.

A “conservative amino acid substitution” involves a substitution of one amino acid residue with another residue such that there is little or no effect on the polarity or charge of the amino acid residue at that position. This is exemplified by the following groups of amino acids, whereby substitutions of one amino acid with a different amino acid in the same group is considered a conservative substitution:
Hydrophilic: Ala, Pro, Gly, Glu, Asp, Gln, Asn, Ser, Thr.
Aliphatic: Val, Ile, Leu, Met.
Basic: Lys, Arg, His.
Aromatic: Phe, Tyr, Trp.

Furthermore, any residue may frequently be substituted with alanine.

Amino acid residue substitutions of an antibody and/or immunoglobulin chain of the present disclosure can be prepared by introducing appropriate nucleotide changes into a nucleic acid of the present disclosure. Such changes include, for example, deletions, insertions or substitutions of residues within the amino acid sequence. A combination of deletion, insertion and substitution can be made to arrive at the final construct, provided that the final polypeptide product possesses the desired characteristics.

As used herein, the term “sequence identity” refers to the relatedness between two amino acid sequences or between two nucleotide sequences is described by the parameter “sequence identity”. For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the-nobrief option must be specified in the command line. The output of Needle labelled “longest identity” is calculated as follows: (Identical Residues×100)/(Length of Alignment−Total Number of Gaps in Alignment). For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NLIC4.4) substitution matrix. In order for the Needle program to report the longest identity, the nobrief option must be specified in the command line. The output of Needle labeled “longest identity” is calculated as follows: (Identical Deoxyribonucleotides×100)/(Length of Alignment−Total Number of Gaps in Alignment)

### Description

### ALK7

The human ALK7 gene (uniprotkb/Q8NER5) encodes a protein consisting of 493 amino acid residues, including a signal peptide, an extra-cellular domain, a transmembrane region and a cytoplasmic domain. The ALK7 protein sequence is:

(SEQ ID NO.: 82)
MTRALCSALRQALLLLAAAAELSPGLKCVCLLCDSSNFTCQTEGACWAS
VMLTNGKEQVIKSCVSLPELNAQVFCHSSNNVTKTECCFTDFCNNITLH
LPTASPNAPKLGPMELAIIITVPVCLLSIAAMLTVWACQGRQCSYRKKK
RPNVEEPLSECNLVNAGKTLKDLIYDVTASGSGSGLPLLVQRTIARTIV
LQEIVGKGRFGEVWHGRWCGEDVAVKIFSSRDERSWFREAEIYQTVMLR
HENILGFIAADNKDNGTWTQLWLVSEYHEQGSLYDYLNRNIVTVAGMIK
LALSIASGLAHLHMEIVGTQGKPAIAHRDIKSKNILVKKCETCAIADLG
LAVKHDSILNTIDIPQNPKVGTKRYMAPEMLDDTMNVNIFESFKRADIY
SVGLVYWEIARRCSVGGIVEEYQLPYYDMVPSDPSIEEMRKVVCDQKFR
PSIPNQWQSCEALRVMGRIMRECWYANGAARLTALRIKKTISQLCVKED
CKA (SEQ ID NO.: 82). In some embodiments, human
ALK7 comprises or consists of the amino acid
sequence according to SEQ ID NO.: 82.

The mature protein is a type I serine/threonine kinases, which forms part of a signalling complex activated by activin B (ActB). The receptor complex is a heterooligomer that consists of two type I (ALK7) and two type II transmembrane serine/threonine kinases. Stimulation by a dimeric ligand, such as ActB, leads to activation of SMAD transcriptional regulators. Inhibition of the signalling complex may be obtained by blocking ligand binding to the receptor, including using an antigen binding protein interacting with the extracellular domain of ALK7.

### Antigen Binding Protein

The present disclosure provides an antigen binding protein binding ALK7. In some embodiments, an antigen binding protein binding ALK7 is an anti-ALK7 antibody or antigen binding fragment thereof. In some embodiments, anti-ALK7 antibody is used interchangeably with ALK7 antibody. In some embodiment, an anti-ALK7 antibody or antigen binding fragment thereof specifically binds the extracellular domain of human ALK7, wherein human ALK7 comprises an amino acid sequence according to SEQ ID NO.: 82. In some embodiments, the extracellular domain (ECD) of ALK7 extends from the signal peptide to the transmembrane region i.e., amino acid residues at position 26-113 of the human ALK7 according to SEQ ID NO.: 82.

In some embodiments, the ECD of ALK7 is shortened at the N-terminal. In some embodiments, a shortened ALK7 ECD comprises amino acids 26-113 and is referred to as ALK7-ECD₂₆₋₁₁₃ herein. In some embodiment, anti-ALK7 antibody or antigen binding fragment thereof binds human ALK7-ECD₂₆₋₁₁₃.

### Antibody and Antibody Fragments

A typical example of an antigen binding protein is an antibody and thus the disclosure provides an antibody binding ALK7, including an isolated antibody binding ALK7 and/or an antibody binding ALK7 specifically. In some embodiments, the present disclosure provides an anti-ALK7 antibody or antigen binding fragment thereof.

In some embodiments, an anti-ALK7 antibody or antigen binding fragment thereof specifically binds to the extracellular domain of human ALK7. In some embodiments, an anti-ALK7 antibody or antigen binding fragment thereof specifically binds to the extracellular domain of human ALK7, wherein the extracellular domain of human ALK7 comprises the amino acid residues 26-113 of SEQ ID NO.: 82. In some embodiments, an anti-ALK7 antibody or antigen binding fragment thereof binds the extracellular domain of ALK7 (amino acid residues 26-113 of SEQ ID NO.: 82). In some embodiments, an anti-ALK7 antibody or antigen binding fragment thereof binds ALK7-ECD (26-113) corresponding to amino acid residues 26-113 of SEQ ID NO. 82).

In some embodiments, an anti-ALK7 antibody or antigen binding fragment thereof comprises two variable regions.

In some embodiments, an anti-ALK7 antibody or antigen binding fragment thereof comprises an antigen binding domain comprising 6 CDR sequences.

In some embodiments, an anti-ALK7 antibody or antigen binding fragment thereof comprises two variable regions each comprising 3 CDR sequences each.

In some embodiments, a variable region(s) comprises several framework sequences.

In some embodiments, a variable region sequence comprises FR1-CDR1-FR2-CDR2-FR3-CDR3-FR4. In some embodiments, FR1-CDR1-FR2-CDR2-FR3-CDR3-FR4 are defined by Kabat and/or IMGT.

In some embodiments, CDRs are defined according to Kabat or IMGT

In some embodiments, CDRs are defined according to Kabat.

In some embodiments, CDRs are defined according to IMGT

In some embodiments, an anti-ALK7 antibody or antigen binding fragment thereof comprises a heavy chain and a light chain.

In some embodiments, an anti-ALK7 antibody or antigen binding fragment thereof comprises a variable heavy chain sequences and a variable light chain sequence.

In some embodiments, an anti-ALK7 antibody or antigen binding fragment thereof comprises a heavy and a light chain variable region each sequence comprising 3 CDRs.

In some embodiments, an anti-ALK7 antibody or antigen binding fragment thereof comprises a heavy chain variable sequence comprising three CDRs and a light chain variable sequence comprising three CDRs.

In some embodiments, the present disclosure provides an anti-ALK7 antibody or antigen binding fragment thereof comprising:
(a) a heavy chain variable region comprising a hCDR1 of SEQ ID NO.: 62 or SEQ ID NO.: 63 with up to 1 amino acid substitution, a hCDR2 of any one of SEQ ID NOs.: 64-70 with up to 1 or 2 amino acid substitutions, and a hCDR3 of SEQ ID NO.: 71, or SEQ ID NO.: 72 with up to 1 or 2 amino acid substitutions, and
(b) a light chain variable region comprising a ICDR1 of any one of SEQ ID NOs.: 73-75 with up to 1 or 2 amino acid substitutions, ICDR2 of SEQ ID NO.: 76 or SEQ ID NO.: 77 with up to 1 amino acid substitutions, and ICDR3 of SEQ ID NO.: 78 or SEQ ID NO.: 79 with up to 1 amino acid substitution.

In some embodiments, an antibody, such as an anti-ALK7 antibody or antigen binding fragment thereof, binding the extracellular domain of human ALK7 (SEQ ID NO.: 82) comprising three heavy chain CDR sequences, hCDR1-3, and three light chain CDR, sequences, ICDR1-3, wherein
a) hCDR1 is SGNYWN (SEQ ID NO.: 62) with up to 1 or 2 amino acid substitutions,
b) hCDR2 is YISFDGRNNYNPSLKN (SEQ ID NO.: 64) with up to 1, 2 or 3 amino acid substitutions,
c) hCDR3 is DYYGSEGFGY (SEQ ID NO.: 71) with up to 1, 2 or 3 amino acid substitutions,
d) ICDR1 is KSSQSLLNSRTRKNYLA (SEQ ID NO.: 73) with up to 1, 2 or 3 amino acid substitutions,
e) ICDR2 is WTSTRES (SEQ ID NO.: 76) with up to 1 or 2 amino acid substitutions, and
f) ICDR3 is KQSYNLPYT (SEQ ID NO.: 78) with up to 1 or 2 amino acid substitutions.

In some embodiments, an antibody, such as an anti-ALK7 antibody or antigen binding fragment thereof, binding the extracellular domain of human ALK7 (SEQ ID NO.: 82) comprising three heavy chain CDR sequences, hCDR1-3, and three light chain CDR, sequences, ICDR1-3, wherein

a) hCDR1 is
(SEQ ID NO.: 62)
SGNYWN
or
(SEQ ID NO.: 63)
SGSYWN
with up to 1 amino acid substitution
b) hCDR2 is
(SEQ ID NO.: 64)
YISFDGRNNYNPSLKN,
(SEQ ID NO.: 65)
YISFDGRTNYNPSLKN,
(SEQ ID NO.: 66)
YISFDGRTNYNPSLKS,
(SEQ ID NO.: 67)
YISFDGRRNYNPSLKN,
(SEQ ID NO.: 68)
YISFTGRTNYNPSLKN,
(SEQ ID NO.: 69)
YISFSGRNNYNPSLKN
or
(SEQ ID NO.: 70)
YISFSGRTNYNPSLKN
with up to 1 or 2 amino acid,
c) hCDR3 is
(SEQ ID NO.: 71)
DYYGSEGFGY
or
(SEQ ID NO.: 72)
DYYGSEGFAY

with up to 1 or 2 amino acid substitutions,
d) ICDR1 is KSSQSLLNSRTRKNYLA (SEQ ID NO.: 73), KSSQSLLVSRTRKNYLA (SEQ ID NO.: 74) or KSSQSLLYSRTRKNYLA (SEQ ID NO.: 75) with up to 1 or 2 amino acid substitutions,
e) ICDR2 is WTSTRES (SEQ ID NO.: 76) or WASTRES (SEQ ID NO.: 77) with up to 1 amino acid substitution, and
f) ICDR3 is KQSYNLPYT (SEQ ID NO.: 78) or KRSYNLPYT (SEQ ID NO.: 79) with up to 1 amino acid substitution.

In some embodiments, an amino acid substitution is a conservative substitution or a substitution with an alanine residue.

In some embodiments, an amino acid substitution is a conservative substitution.

In some embodiments, an amino acid substitution is a substitution with an alanine residue.

In some embodiments, the present disclosure provides an anti-ALK7 antibody or antigen binding fragment thereof comprising:
(a) a heavy chain variable region comprising a hCDR1 of SEQ ID NO.: 62 or SEQ ID NO.: 63, a hCDR2 of any one of SEQ ID NOs.: 64-70, and a hCDR3 of SEQ ID NO.: 71, or SEQ ID NO.: 72, and
(b) a light chain variable region comprising a ICDR1 of any one of SEQ ID NOs.: 73-75, ICDR2 of SEQ ID NO.: 76 or SEQ ID NO.: 77, and ICDR3 of SEQ ID NO.: 78 or SEQ ID NO.: 79.

In some embodiments, the present disclosure provides an anti-ALK7 antibody or antigen binding fragment thereof comprising:
(a) a heavy chain variable region comprising a hCDR1 of SEQ ID NO.: 63, a hCDR2 of SEQ ID NO.: 65, and a hCDR3 of SEQ ID NO.: 72, and
(b) a light chain variable region comprising a ICDR1 of SEQ ID NO.: 74, ICDR2 of SEQ ID NO.: 76, and ICDR3 of SEQ ID NO.: 78.

In some embodiments, the disclosure provides an antibody, such as an anti-ALK7 antibody or antigen binding fragment thereof, binding the extracellular domain of human ALK7 (SEQ ID NO.: 82) and comprising three heavy chain CDR sequences, hCDR1-3, and three light chain CDR, sequences, ICDR1-3, wherein

a) hCDR1 is
(SEQ ID NO.: 62)
SGNYWN
or
(SEQ ID NO.: 63)
SGSYWN,
b) hCDR2 is
(SEQ ID NO.: 64)
YISFDGRNNYNPSLKN,
(SEQ ID NO.: 65)
YISFDGRTNYNPSLKN,
(SEQ ID NO.: 66)
YISFDGRTNYNPSLKS,
(SEQ ID NO.: 67)
YISFDGRRNYNPSLKN,
(SEQ ID NO.: 68)
YISFTGRTNYNPSLKN,
(SEQ ID NO.: 69)
YISFSGRNNYNPSLKN
or
(SEQ ID NO.: 70)
YISFSGRTNYNPSLKN
c) hCDR3 is
(SEQ ID NO.: 71)
DYYGSEGFGY
or
(SEQ ID NO.: 72)
DYYGSEGFAY,
d) ICDR1 is
(SEQ ID NO.: 73)
KSSQSLLNSRTRKNYLA,
(SEQ ID NO.: 74)
KSSQSLLVSRTRKNYLA,
or
(SEQ ID NO.: 75)
KSSQSLLYSRTRKNYLA,
e) ICDR2 is
(SEQ ID NO.: 76)
WTSTRES
or
(SEQ ID NO.: 77)
WASTRES
and
f) ICDR3 is
(SEQ ID NO.: 78)
KQSYNLPYT
or
(SEQ ID NO.: 79)
KRSYNLPYT.

In some embodiments, the present disclosure provides an anti-ALK7 antibody or antigen binding fragment thereof comprises:
(a) a heavy chain variable region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to any one of SEQ ID NO.: 1-34, and
(b) a light chain variable region comprising an amino acid sequence that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% identical to any one of SEQ ID NO.: 35-59.

In some embodiments, the present disclosure provides an anti-ALK7 antibody or antigen binding fragment thereof comprises:
(a) a heavy chain variable region comprising an amino acid sequence according to any one of SEQ ID NO.: 1-34 with up to 5 amino acid substitutions, such as up to 4 amino acid substitutions, such as up to 4 amino acid substitutions, such as up to 2 amino acid substitutions, such as up to 1 amino acid substitution, and
(b) a light chain variable region comprising an amino acid sequence according to any one of SEQ ID NO.: 35-59 with up to 5 amino acid substitutions, such as up to 4 amino acid substitutions, such as up to 4 amino acid substitutions, such as up to 2 amino acid substitutions, such as up to 1 amino acid substitution.

In some embodiments, the present disclosure provides an anti-ALK7 antibody or antigen binding fragment thereof comprises:
(a) a heavy chain variable region comprising an amino acid sequence according to any one of SEQ ID NO.: 1-34, and
(b) a light chain variable region comprising an amino acid sequence according to any one of SEQ ID NO.: 35-59.

In some embodiments, the present disclosure provides an antibody, such as an anti-ALK7 antibody or antigen binding fragment thereof, binding the extracellular domain of human ALK7 (SEQ ID NO.: 82) and comprising three heavy chain CDR sequences, hCDR1-3, and three light chain CDR, sequences, ICDR1-3, wherein
i. the three heavy chain CDR sequences are selected from the CDR sequences of any one of the heavy chain variable regions (HV) identified by SEQ ID NO.: 1-34.
ii. the three light chain CDR sequences are selected from the CDR sequences of any one of the light chain variable regions (LV) identified by SEQ ID NO.: 35-59.

In some embodiments, an anti-ALK7 antibody or antigen binding fragment thereof comprises:
(a) a heavy chain variable region comprising an amino acid sequence of SEQ ID NO.: 18, and
(b) a light chain variable region comprising an amino acid sequence of SEQ ID NO.: 53.

In some embodiments, an antibody is an antibody or an antigen binding fragment thereof.

In some embodiments, an anti-ALK7 antibody or antigen binding fragment thereof is or comprises a Fab, a Fab′, a F(ab′)₂, a Fv, a Fd, a dAb, an scFv, or a single domain antibody.

In some embodiments, an anti-ALK7 antibody or antigen binding fragment thereof is a full-length antibody. In some embodiments, an anti-ALK7 antibody or antigen binding fragment thereof is an IgG antibody, such as an IgG1, IgG2, IgG3, or IgG4 antibody.

In some embodiments, an anti-ALK7 antibody or antigen binding fragment thereof comprises heavy and light chain constant regions of an IgG sub type, such as any of IgG1, IgG2, IgG3, or IgG4.

In some embodiments, a heavy chain constant region comprises an amino acid sequence of ASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPEAEGAPS VFLFPPKPKDTLMISRTPEVTCVVVDVSHEDPEVKFNWYVDGVEVHNAKTKPREEQYNSTY RVVSVLTVLHQDWLNGKEYKCKVSNKALPSSIEKTISKAKGQPREPQVYTLPPSREEMTKN QVSLTCLVKGFYPSDIAVEWESNGQPENNYKTTPPVLDSDGSFFLYSKLTVDKSRWQQGN VFSCSVMHEALHNHYTQKSLSLSPGK (SEQ ID NO.: 60). In some embodiments, a heavy chain constant region comprises an amino acid sequence having a sequence identify that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% to SEQ ID NO.: 60. In some embodiments, a heavy chain constant region comprises an amino acid sequence of SEQ ID NO.: 60 with up to 5 amino acid substitutions, such as up to 4 amino acid substitutions, such as up to 4 amino acid substitutions, such as up to 2 amino acid substitutions, such as up to 1 amino acid substitution. In some embodiments, a heavy chain constant region comprises an amino acid sequence of SEQ ID NO.: 60, wherein the C-terminal lysine (K) is optional.

In some embodiments, a heavy chain constant region comprises an amino acid sequence of ASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPEAEGAPS VFLFPPKPKDTLMISRTPEVTCVVVDVSHEDPCVKFNWYVDGVEVHNAKTKPCEEQYNSTY RCVSVLTVLHQDWLNGKEYKCKVSNKALPSSIEKTISKAKGQPREPQVYTLPPSREEMTKN QVSLTCLVKGFYPSDIAVEWESNGQPENNYKTTPPVLDSDGSFFLYSKLTVDKSRWQQGN VFSCSVMHEALHNHYTQKSLSLSPG (SEQ ID NO.: 87). In some embodiments, a heavy chain constant region comprises an amino acid sequence having a sequence identify that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% to SEQ ID NO.: 87. In some embodiments, a heavy chain constant region comprises an amino acid sequence of SEQ ID NO.: 87 with up to 5 amino acid substitutions, such as up to 4 amino acid substitutions, such as up to 4 amino acid substitutions, such as up to 2 amino acid substitutions, such as up to 1 amino acid substitution. In some embodiments, a heavy chain constant region comprises an amino acid sequence of SEQ ID NO.: 87, wherein the C-terminal lysine (K) is optional.

In some embodiments, a heavy chain constant region comprises an amino acid sequence of RTVAAPSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO: 61). In some embodiments, a light chain constant region comprises an amino acid sequence having a sequence identify that is at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% to SEQ ID NO.: 61. In some embodiments, a light chain constant region comprises an amino acid sequence of SEQ ID NO.: 61 with up to 5 amino acid substitutions, such as up to 4 amino acid substitutions, such as up to 4 amino acid substitutions, such as up to 2 amino acid substitutions, such as up to 1 amino acid substitution. In some embodiments, a light chain constant region comprises an amino acid sequence of SEQ ID NO.: 61.

In some embodiments, an anti-ALK7 antibody comprises heavy and light chain constant regions including one or more amino acid substitutions to optimise Fc function.

This may include amino acid substitutions affecting cell interaction or effector protein interactions, such as FcR effector function or complement system effector functions, such as mutations equivalent to L117A, L118E, G120A, A213S and P214S in SEQ ID NO.: 60 or SEQ ID NO: 87.

Using the EU numbering system, the mutations are referred to as L234A, L235E, G237A, A330S, and P331S. In some embodiments, a constant region comprises one or more of L234A, L235E, G237A, A330S, P331S, or combinations thereof.

### Affinity

The affinity of an antigen binding protein to its antigen can be determined by various methods, such as Surface Plasmon Resonance (SPR) wherein the kinetics of complex formation and dissociation is measures. An example of such an assay is provided in Example 3 herein, where binding of ALK7 to the captured test antibodies was examined by injecting the analyte (including ALK7) over both flow cells, enabling comparative analyses of the target binding to the captured antibodies relative to the reference surface. As described herein the provided anti-ALK7 antibodies have a high affinity when measured by SPR.

In some embodiments, the affinity (K_{D}) for an antigen binding protein, such as an anti-ALK7 antibody, is 1-1000 nM, such as 10-750 nM, or such as 10-500 nM.

In some embodiments, the affinity (K_{D}) for an antigen binding protein, such as an anti-ALK7 antibody, is below 10 μM, such as below 5 μM, such as below 1 μM.

In some embodiments, the affinity (K_{D}) for an antigen binding protein, such as an anti-ALK7 antibody, is below 100 nM, such as below 50 nM, such as below 10 nM.

### ALK7 Inhibition/Potency

The binding affinity as measured by SPR describes the monomeric interaction between one ligand and one binding protein in a controlled environment. In vivo, the valency of the binding and diffusional fluctuations may strongly affect the macroscopic binding affinity. In order to further evaluate the functionality of the antigen binding proteins a cellular assay has been used. As described in Examples 4, a reporter gene assay (RGA) was used herein to determine the ability of an anti-ALK7 antibody to inhibit ALK7 signaling.

ALK7 signaling can be measured by placing a luciferase gene under control of a SMAD binding element (SBE). Since stimulation of ALK7 is known to phosphorylate SMAD3, which binds SBE, ALK7 stimulation results in increased luciferase activity.

The assay was used to identify antibodies inhibiting ALK7 signaling and the (inhibitory) potency of the antibody and selected variants thereof is provided in Table 4.1 in the examples.

In some embodiments, an anti-ALK7 antibody or antigen binding fragment thereof inhibits ALK7 signalling in the RGA assay of Example 4. In one embodiment the IC50 is below 10 μM, such as below 5 μM, such as below 1 μM. In one embodiment the IC50 is below 100 nM, such as below 50 nM, such as below 10 nM. In one embodiment the IC50 is below 40 nM, such as below 25 nM, such as below 8 nM.

Alternatively, ALK7 inhibition may be measure in a SMAD3 signalling assay as described in Example 4. The assay was used to characterize antibodies inhibiting ALK7 signaling and the (inhibitory) potency of selected antibody variants identified is provided in Table 1.2 in the examples.

In some embodiments, an anti-ALK7 antibody or antigen binding fragment thereof inhibits SMAD3 signalling in the SMAD3 assay of Example 4. In one embodiment the IC50 is below 10 μM, such as below 5 μM, such as below 1 μM. In one embodiment the IC50 is below 100 nM, such as below 50 nM, such as below 10 nM. In one embodiment the IC50 is below 40 nM, such as below 25 nM, such as below 8 nM.

### Binding Specificity and Epitope

In some embodiments, anti-ALK7 antibodies or antigen binding fragments thereof bind to a particular ALK7 epitope.

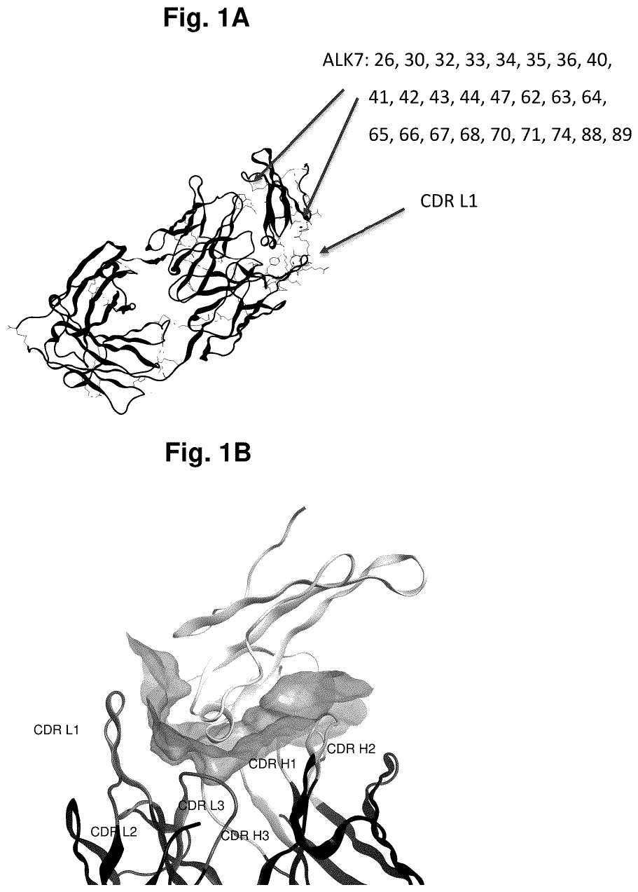

In some embodiments, anti-ALK7 antibodies or antigen binding fragments thereof as provided herein have a CDR L1 that shapes the paratope and confer steric constrains to the antibody:ALK7interface by contacts to turns in ALK7. In some embodiments, an anti-ALK7 antibody CDR H2 makes interactions with a groove in ALK7, demonstrating a highly convex epitope (FIG. 1).

As further described, the epitope is determined as the amino acid residues within 5 Å of an anti-ALK7 antibody in the crystal structure. In some embodiments, the epitope includes several individual amino acid stretches separated by a region spanning amino acid residues 48-61. In some embodiments, the region spanning amino acid residues 48-61 is not in close contact with the antigen binding region of the antibody, such as the anti-ALK7 antibody. In some embodiments, the region spanning amino acid residues 48-61 is not within 5 Å from an antigen binding region of the antibody.

In some embodiments, an anti-ALK7 antibody or antigen binding fragment thereof binds at least amino acid residues 26 to 47 of human ALK7 of SEQ ID NO.: 82. In some embodiments, an anti-ALK7 antibody or antigen binding fragment thereof binds at least amino acid residues 62 to 74 of human ALK7 of SEQ ID NO.: 82. In some embodiments, an anti-ALK7 antibody or antigen binding fragment thereof binds at least amino acid residues 62 to 88 of human ALK7 of SEQ ID NO.: 82. In some embodiments, an anti-ALK7 antibody or antigen binding fragment thereof binds at least amino acid residues 48 to 61 of human ALK7 of SEQ ID NO.: 82. In some embodiments, an anti-ALK7 antibody or antigen binding fragment thereof binds at least amino acid residues 75 to 87 of human ALK7 of SEQ ID NO.: 82.

In some embodiments, an anti-ALK7 antibody or antigen binding fragment thereof binds a non-contiguous epitope that comprises amino acid residues 26 to 47 and amino acid residues 62 to 74 of ALK7.

In some embodiments, an anti-ALK7 antibody or antigen binding fragment thereof binds a non-contiguous epitope that comprises amino acid residues 26 to 47 and amino acid residues 62 to 88 of ALK7.

In some embodiments, an anti-ALK7 antibody or antigen binding fragment thereof binds a non-contiguous epitope that comprises amino acid residues 26-47, amino acid residues 62-74, and amino acid residue 88 of ALK7.

In some embodiments, an anti-ALK7 antibody or antigen binding fragment thereof binds an epitope that does not include amino acid residue within amino acid residues 48 to 61 of ALK7.

In some embodiments, an anti-ALK7 antibody or antigen binding fragment thereof binds an epitope that does not include amino acid residue within amino acid residues 75 to 87 of ALK7.

In some embodiments, an epitope is defined as amino acid residues within 5 Å of the antibody as determined by X-ray diffraction.

In some embodiments, an anti-ALK7 antibody or antigen binding fragment thereof binds an epitope comprising one or more amino acid residues selected from the group consisting of L26, C30, L32, C33, S35, S36, C40, Q41, T42, E43, G44, W47, S62, C63, V64, S65, L66, P67, E68, N70, A71, F74, and F88 of human ALK7 (SEQ ID NO.: 82).

In some embodiments, an anti-ALK7 antibody or antigen binding fragment thereof binds an epitope comprising two, three, four or more of amino acid residues selected from the group consisting of L26, C30, L32, C33, S35, S36, C40, Q41, T42, E43, G44, W47, S62, C63, V64, S65, L66, P67, E68, N70, A71, F74, and F88 of human ALK7 (SEQ ID NO.: 82).

In some embodiments, an anti-ALK7 antibody or antigen binding fragment thereof binds an epitope comprising five, six, seven or more amino acid residues selected from the group consisting of L26, C30, L32, C33, S35, S36, C40, Q41, T42, E43, G44, W47, S62, C63, V64, S65, L66, P67, E68, N70, A71, F74, and F88 of human ALK7 (SEQ ID NO.: 82).

In some embodiments, an anti-ALK7 antibody or antigen binding fragment thereof binds an epitope comprising eight, nine, ten or more amino acid residues selected from the group consisting of L26, C30, L32, C33, S35, S36, C40, Q41, T42, E43, G44, W47, S62, C63, V64, S65, L66, P67, E68, N70, A71, F74, and F88 of human ALK7 (SEQ ID NO.: 82).

In some embodiments, an anti-ALK7 antibody or antigen binding fragment thereof binds an epitope comprising
i. at least two amino acid residues selected from L26, C30, L32, C33, S35, S36, C40, Q41, T42, E43, G44, or W47,
ii. at least two amino acid residues selected from S62, C63, V64, S65, L66, P67, E68, N70, A71, or F74, and
iii. at least one amino acid residue of F88 and T89;
wherein amino acid residues are numbered according to human ALK7 of SEQ ID NO.: 82.

In some embodiments, an anti-ALK7 antibody or antigen binding fragment thereof binds an epitope comprising
i. an amino acid residue L26,
ii. at least three amino acid residue selected from C30, L32, C33, S35, S36, C40, Q41, T42, E43, G44, or W47,
iii. at least three amino acid residue selected from S62, C63, V64, S65, L66, P67, E68, N70, A71, or F74, and
iv. at least one amino acid residue of F88 and 89;
wherein amino acid residues are numbered according to ALK7 of SEQ ID NO.: 82.

### Method of Producing an ALK7 Binding Protein.

The present disclosure also provides methods of producing an anti-ALK7 antibody or antigen binding fragment thereof. The methods may entail cloning of an antibody sequence in a suitable expression vector and establishing a host cell expressing such antibody.

The present disclosure also provides a nucleotide sequence or pair of nucleotide sequences encoding an anti-ALK7 antibody or antigen binding fragment thereof as described herein.

The present disclosure also provides a host cell comprising a nucleotide sequence or pair of nucleotide sequences encoding anti-ALK7 antibody or antigen binding fragment thereof as described herein.

The present disclosure also provides a host cell expressing an anti-ALK7 antibody or antigen binding fragment thereof as described herein.

The present disclosure also provides a method of producing an anti-ALK7 antibody or antigen binding fragment thereof comprising the steps of:
a) culturing a host cell under conditions conducive of expressing an anti-ALK7 antibody or antigen binding fragment thereof as provided herein; and
b) purifying said anti-ALK7 antibody or antigen binding fragment thereof.

### Pharmaceutical Composition

The present disclosure further provides a pharmaceutical composition comprising an anti-ALK7 antibody or antigen binding fragment thereof as described herein.

In some embodiments, a pharmaceutical composition comprises an anti-ALK7 antibody or antigen binding fragment thereof as described herein and one or more pharmaceutical excipients, such as a buffer system, tonicity agent(s), chelating agent(s), stabilizer(s), surfactant(s) and preservative(s).

For convenience reference is made to Remington: The Science and Practice of Pharmacy, 20^{th} edition, 2000.

In some embodiments, a pharmaceutical composition is a liquid formulation. In some embodiments, a pharmaceutical composition is an aqueous formulation. In some embodiments, a formulation may be obtained by solubilization freeze dried material, such as freeze dried anti-ALK7 antibodies or antigen binding fragments thereof.

### Therapeutic Uses

The present disclosure provides medical use of an anti-ALK7 antibody or antigen binding fragment thereof described herein. The antibodies are useful in treating, ameliorating, and/or preventing diseases or disorders where inhibition of ALK7 signalling is beneficial. Such diseases and disorders include diseases and disorders related to metabolism, including type 2 diabetes, overweight, obesity, other metabolic diseases or conditions, and cardiovascular disease (CVD).

In some embodiments, the disclosure provides use of an anti-ALK7 antibody or antigen binding fragment thereof as provided herein as a medicament or in a method of treatment or therapy.

In some embodiments, anti-ALK7 antibody or antigen binding fragment thereof provided herein are for use in a method of treatment of disorders including diseases and disorders related to metabolism, such as type 2 diabetes and/or obesity. In some embodiments, an anti-ALK7 antibody or antigen binding fragment thereof provided herein are for use in a method of treating and/or preventing type 2 diabetes or obesity. In some embodiments, an anti-ALK7 antibody or antigen binding fragment thereof provided herein are for use in weight management of a human subject in need thereof. In some embodiments, weight management is the treatment of obesity or overweight. In some embodiments, wherein for the treatment of overweight said subject may have at least one weight-related co-morbidity.

In some embodiments, a method provided herein comprises administering anti-ALK7 antibody or antigen binding fragment thereof to a subject. The subject may be a human subject in need of such treatment, such as a patient suffering from a disease or disorder mentioned above.

Antibodies provided herein are further useful in methods of reducing weight or inducing weight loss in a subject, such as a human subject.

In some embodiments, a method as provided herein includes administration of a therapeutically effective dosage of an anti-ALK7 antibody or antigen binding fragment thereof provided herein. The dosage and frequency of administration of an ALK7 antibody or antigen binding fragment thereof might depend on the individual subject and the disease or disorder to be treated.

The medical definition of obesity may differ from country to country, but generally individuals with a BMI above 25 are considered overweight (or pre-obese), and individuals with a BMI above 30 are considered obese, while some Asian guidelines do not discrimination between overweight and obesity.

In some embodiments, methods provided herein include administration of an anti-ALK7 antibody or antigen binding fragment thereof to a human with a BMI above 25, such as a BMI above 26, 27, 28, 29 or 30. In some embodiments, methods provided herein comprises administration of an anti-ALK7 antibody or antigen binding fragment thereof to a human with a BMI above 30, such as a BMI above 31, 32, 33, 34, or 35.

### EMBODIMENTS

1. An antibody binding the extracellular domain of human ALK7 (SEQ ID NO.: 82) comprising three heavy chain CDR sequences, hCDR1-3, and three light chain CDR, sequences, ICDR1-3, wherein
a) hCDR1 is SGNYWN (SEQ ID NO.: 62) or SGSYWN (SEQ ID NO.: 63) with up to 1 amino acid substitutions,
b) hCDR2 is hCDR2 is YISFDGRNNYNPSLKN (SEQ ID NO.: 64), YISFDGRTNYNPSLKN (SEQ ID NO.: 65), YISFDGRTNYNPSLKS (SEQ ID NO.: 66), YISFDGRRNYNPSLKN (SEQ ID NO.: 67), YISFTGRTNYNPSLKN (SEQ ID NO.: 68), YISFSGRNNYNPSLKN (SEQ ID NO.: 69) or YISFSGRTNYNPSLKN (SEQ ID NO.: 70) with up to 1 or 2 amino acid,
c) hCDR3 is DYYGSEGFGY (SEQ ID NO.: 71) or DYYGSEGFAY (SEQ ID NO.: 72) with up to 1 or 2 amino acid substitutions,
d) ICDR1 is KSSQSLLNSRTRKNYLA (SEQ ID NO.: 73), KSSQSLLVSRTRKNYLA (SEQ ID NO.: 74) or KSSQSLLYSRTRKNYLA (SEQ ID NO.: 75) with up to 1 or 2 amino acid substitutions,
e) ICDR2 is WTSTRES (SEQ ID NO.: 76) or WASTRES (SEQ ID NO.: 77) with up to 1 amino acid substitutions and
f) ICDR3 is KQSYNLPYT (SEQ ID NO.: 78) or KRSYNLPYT (SEQ ID NO.: 79) with up to 1 amino acid substitutions.

2. An antibody according to embodiment 1, wherein
a) hCDR1 is SGSYWN (SEQ ID NO.: 63) with up to 1 or 2 amino acid substitutions,
b) hCDR2 is YISFDGRTNYNPSLKN (SEQ ID NO.: 65) with up to 1, 2 or 3 amino acid substitutions,
c) hCDR3 is DYYGSEGFAY (SEQ ID NO.: 72) with up to 1, 2 or 3 amino acid substitutions,
d) ICDR1 is KSSQSLLVSRTRKNYLA (SEQ ID NO.: 74) with up to 1, 2 or 3 amino acid substitutions, and
e) ICDR2 is WTSTRES (SEQ ID NO.: 76) with up to1 or 2 amino acid substitutions,
f) ICDR3 is KQSYNLPYT (SEQ ID NO.: 78) with up to 1 or 2 amino acid substitutions.

3. The antibody according to any of the previous embodiments, wherein

a) hCDR1 is
(SEQ ID NO.: 62)
SGNYWN
or
(SEQ ID NO.: 63)
SGSYWN,
b) hCDR2 is
(SEQ ID NO.: 64)
YISFDGRNNYNPSLKN,
(SEQ ID NO.: 65)
YISFDGRTNYNPSLKN,
(SEQ ID NO.: 66)
YISFDGRTNYNPSLKS,
(SEQ ID NO.: 67)
YISFDGRRNYNPSLKN,
(SEQ ID NO.: 68)
YISFTGRTNYNPSLKN,
(SEQ ID NO.: 69)
YISFSGRNNYNPSLKN
or
(SEQ ID NO.: 70)
YISFSGRTNYNPSLKN,
c) hCDR3 is
(SEQ ID NO.: 71)
DYYGSEGFGY
or
(SEQ ID NO.: 72)
DYYGSEGFAY,
d) ICDR1 is
(SEQ ID NO.: 73)
KSSQSLLNSRTRKNYLA,
(SEQ ID NO.: 74)
KSSQSLLVSRTRKNYLA
or
(SEQ ID NO.: 75)
KSSQSLLYSRTRKNYLA,
e) ICDR2 is
(SEQ ID NO.: 76)
WTSTRES
or
(SEQ ID NO.: 77)
WASTRES
and
f) ICDR3 is
(SEQ ID NO.: 78)
KQSYNLPYT
or
(SEQ ID NO.: 79)
KRSYNLPYT.

4. The antibody according to any of the previous embodiments, wherein

a) hCDR1 is
(SEQ ID NO.: 63)
SGSYWN,
b) hCDR2 is
(SEQ ID NO.: 65)
YISFDGRTNYNPSLKN,
c) hCDR3 is
(SEQ ID NO.: 71)
DYYGSEGFGY,
d) ICDR1 is
(SEQ ID NO.: 73)
KSSQSLLNSRTRKNYLA
or
(SEQ ID NO.: 74)
KSSQSLLVSRTRKNYLA,
e) ICDR2 is
(SEQ ID NO.: 76)
WTSTRES,
and
f) ICDR3 is
(SEQ ID NO.: 78)
KQSYNLPYT.

5. An antibody binding the extracellular domain of human ALK7 (SEQ ID NO.: 82) comprising three heavy chain CDR sequences and three light chain CDR sequences, wherein
i. the three heavy chain CDR sequences are selected from the CDR sequences of any one of the heavy chain variable regions (HV) identified by SEQ ID NO.: 1-34 and
ii. the three light chain CDR sequences are selected from the CDR sequences of any one of the light chain variable region (LV) identified by SEQ ID NO.: 35-59.

6. The antibody according to any of the previous embodiments, wherein the three heavy chain CDR sequences and the three light chain CDR sequences are selected from the CDR sequences from one of the antibody binding region described in Table 2.1.

7. The antibody according to any of the previous embodiments, wherein the six CDRs are selected from the CDRs of the pairs of heavy and light chain variable region sequences provided in 2.1.

8. The antibody according to any of the previous embodiments, wherein the three heavy chain CDR sequences and the three light chain CDR sequences are selected from the CDR sequences from one of the antibodies V23, V24, V25 and V36.

9. The antibody according to any of the previous embodiments, wherein the CDRs are defined according to kabat or IMGT.

10. The antibody according to any of the previous embodiments, wherein
a) the heavy chain variable region is selected from SEQ ID NOs: 1-34 and
b) the light chain variable region is selected from SEQ ID NOs: 35-59.

11. The antibody according to any of the previous embodiments, wherein
a) the heavy chain variable region is selected from SEQ ID NOs: 3-34 and
b) the light chain variable region is selected from SEQ ID NOs: 38-59.

12. The antibody according to any of the previous embodiments, wherein the light chain and heavy chain variable regions are selected from the sequence pairs of Table 2.1.

13. The antibody according to any of the previous embodiments, wherein the light chain and heavy chain variable regions are selected from the sequence pairs for V1-V50 defined in the table below

| | Heavy Chain Variable | Light Chain Variable |
|---|---|---|
| mAb | region (SEQ ID No.:) | region (SEQ ID No.:) |
| V1 | 3 | 38 |
| V2 | 4 | 39 |
| V3 | 4 | 40 |
| V4 | 5 | 41 |
| V5 | 6 | 41 |
| V6 | 7 | 42 |
| V7 | 8 | 43 |
| V8 | 9 | 44 |
| V9 | 9 | 45 |
| V10 | 10 | 39 |
| V11 | 11 | 39 |
| V12 | 12 | 46 |
| V13 | 13 | 47 |
| V14 | 13 | 48 |
| V15 | 13 | 49 |
| V16 | 13 | 50 |
| V17 | 14 | 50 |
| V18 | 15 | 50 |
| V19 | 16 | 51 |
| V20 | 17 | 51 |
| V21 | 18 | 47 |
| V22 | 19 | 52 |
| V23 | 18 | 53 |
| V24 | 8 | 54 |
| V25 | 8 | 55 |
| V26 | 20 | 55 |
| V27 | 8 | 56 |
| V28 | 21 | 52 |
| V29 | 22 | 52 |
| V30 | 23 | 57 |
| V31 | 22 | 57 |
| V32 | 24 | 57 |
| V33 | 25 | 57 |
| V34 | 26 | 57 |
| V35 | 27 | 53 |
| V36 | 26 | 53 |
| V37 | 28 | 58 |
| V38 | 22 | 58 |
| V39 | 25 | 58 |
| V40 | 29 | 59 |
| V41 | 30 | 59 |
| V42 | 23 | 59 |
| V43 | 20 | 59 |
| V44 | 25 | 59 |
| V45 | 18 | 53 |
| V46 | 26 | 53 |
| V47 | 31 | 53 |
| V48 | 32 | 53 |
| V49 | 33 | 47 |
| V50 | 34 | 57 |

14. An antibody binding the extracellular domain of human ALK7 (SEQ ID NO.: 82), wherein the antibody binds a non-contiguous epitope.

15. The antibody according to any of the previous embodiments, wherein the antibody binds a non-contiguous epitope including aa 26 to 47 and aa 62 to 74 of ALK7, respectively.

16. The antibody according to any of the previous embodiments, wherein the antibody binds a non-contiguous epitope comprising including aa 26-47, aa 62-74 and aa 88-89 of ALK7.

17. The antibody according to any of the previous embodiments, wherein the antibody binds a non-contiguous epitope which does not include any amino acid residues within aa 48 to aa 61 of ALK7.

18. The antibody according to any of the previous embodiments, wherein the antibody binds an epitope comprising one or more of L26, C30, L32, C33, S35, S36, C40, Q41, T42, E43, G44, W47, S62, C63, V64, S65, L66, P67, E68, N70, A71, F74 and F88.

19. The antibody according to any of the previous embodiments, wherein the antibody binds an epitope comprising two, three, four or more of L26, C30, L32, C33, S35, S36, C40, Q41, T42, E43, G44, W47, S62, C63, V64, S65, L66, P67, E68, N70, A71, F74 and F88.

20. The antibody according to any of the previous embodiments, wherein the antibody binds an epitope comprising five, six, seven or more of L26, C30, L32, C33, S35, S36, C40, Q41, T42, E43, G44, W47, S62, C63, V64, S65, L66, P67, E68, N70, A71, F74 and F88.

21. The antibody according to any of the previous embodiments, wherein the antibody binds an epitope comprising eight, nine, ten or more of L26, C30, L32, C33, S35, S36, C40, Q41, T42, E43, G44, W47, S62, C63, V64, S65, L66, P67, E68, N70, A71, F74 and F88.

22. The antibody according to any of the previous embodiments, wherein the antibody binds an epitope comprising
i. at least two of L26, C30, L32, C33, S35, S36, C40, Q41, T42, E43, G44, W47,
ii. at least two of S62, C63, V64, S65, L66, P67, E68, N70, A71, F74, and
iii. at least one of F88 and T89.

23. The antibody according to any of the previous embodiments, wherein the antibody binds an epitope comprising
i. at least two of C30, L32, C33, S35, S36, C40, Q41, T42, E43, G44, W47,
ii. at least two of S62, C63, V64, S65, L66, P67, E68, N70, A71, F74, and
iii. at least one of F88 and T89.

24. The antibody according to any of the previous embodiments, wherein the antibody binds an epitope comprising
i. L26,
ii. at least three of C30, L32, C33, S35, S36, C40, Q41, T42, E43, G44, W47,
iii. at least three of S62, C63, V64, S65, L66, P67, E68, N70, A71, F74, and
iv. at least one of F88 and T89.

25. The antibody according to any of the previous embodiments wherein the epitope is defined as amino acid residues within 5 Å of the antibody as determined by X-ray diffraction.

26. The antibody according to any of the previous embodiments, wherein the antibody preferentially binds an ALK7 dimeric complex.

27. The antibody according to any of the previous embodiments, wherein the antibody binds to an ALK7 dimeric complex with higher affinity than to monomeric ALK7.

28. The antibody according to any of the previous embodiments, wherein the antibody binds to an ALK7 dimeric complex with higher avidity than to monomeric ALK7.

29. The antibody according to any of the previous embodiments, wherein the antibody the is bivalent.

30. The antibody according to any of the previous embodiments, wherein the antibody comprises two identical binding domains.

31. The antibody according to any of the previous embodiments, wherein the affinity (K_{D}) to ALK7 is 1-1000 nM, such as 10-750 nM, or such as 10-500 nM when measured by SPR as in Example 3 herein.

32. The antibody according to any of the previous embodiments, wherein the antibody
a) antagonizes the ALK7 receptor,
b) inhibits ALK7 signalling,
c) inhibits SMAD2/3 activation, and/or
d) inhibits a ligand, such as activin B, binding to the ALK7 receptor.

33. The antibody according to any of the previous embodiments, wherein the antibody inhibits ALK7 signalling.

34. The antibody according to embodiment 33, wherein the IC50 is below 50 nM, such as below 10 nM when measured in the reporter gene assay (RGA) of Example 4.

35. The antibody according to any of the previous embodiments, wherein the antibody binds the extracellular domain of human ALK7 (ALK7-ECD₂₂₋₁₁₃) defined by amino acid 22-113 of SEQ ID NO.: 82.

36. The antibody according to any of the previous embodiments, wherein the antibody binds the extracellular domain of human ALK7 (ALK7-ECD₂₆₋₁₁₃) defined by amino acid 26-113 of SEQ ID NO.: 82.

37. The antibody according to any of the previous embodiments, wherein the antibody comprises light chain constant region and a heavy chain constant region.

38. The antibody according to any of the previous embodiments, wherein the antibody comprises a light chain constant region and a heavy chain constant region of the IgG type, such as the IgG1, IgG2, IgG3, or IgG4.

39. The antibody according to any of the previous embodiments, wherein the antibody comprises a light chain constant region and a heavy chain constant region comprising one or more amino acid substitutions.

40. The antibody according to any of the previous embodiments, wherein the antibody comprises one or more amino acid substitution affecting cell interaction or effector protein interactions.

41. The antibody according to any of the previous embodiments, wherein the antibody comprises one or more amino acid substitution reducing FcR effector function or complement system effector functions.

42. The antibody according to any of the previous embodiments, wherein the antibody comprises one or more amino acid substitution(s) selected from L234A, L235E, G237A, A330S, and P331S (EU numbering).

43. The antibody according to any of the previous embodiments, wherein the antibody comprises a heavy chain constant region of SEQ ID NO.: 60 or SEQ ID NO: 87.

44. The antibody according to any of the previous embodiments, wherein the antibody comprises a light chain constant region of SEQ ID NO.: 61.

45. The antibody according to any of the previous embodiments, wherein the antibody comprises one or more amino acid substitution equivalent to L117A, L118E, G120A, A213S, and P214S in SEQ ID NO.: 60 or SEQ ID NO: 86.

46. The antibody according to any of the previous embodiments, wherein the heavy chain constant region is SEQ ID NO.: 60 or SEQ ID NO: 86 with the amino acid substitution L117A, L118E, G120A, A213S, and P214S.

47. The antibody according to any of the previous embodiments, wherein the antibody is a full-length antibody.

48. The antibody according to any of the previous embodiments, wherein the antibody is an antigen binding fragment of an antibody.

49. The antibody according to any of the previous embodiments, wherein the antibody is an antigen binding fragment comprising a CH1 domain.

50. The antibody according to one any of the previous embodiments, wherein the antibody fragment is selected from a group consisting of: Fab, Fab′, F(ab)2, F(ab′)2, F(ab)S, Fv, single-chain Fv (scFv), dsFv, Fd, and dAb.

51. An antibody according to any one of embodiments 1-50 for use as a medicament.

52. An antibody according to any one of embodiments 1-50 for use in a method of treatment or therapy.

53. An antibody according to any one of embodiments 1-50 for use in a method of treatment of a metabolic disease or disorder.

54. An antibody according to any one of embodiments 1-50 for use in a method of treatment of type 2 diabetes or obesity.

55. An antibody according to any one of embodiments 1-50 for use in a method of treatment of overweight or obesity.

56. An antibody according to any one of embodiments 1-50 for use in a method of treatment to reduce body weight.

57. A method for reducing body weight comprising administering of an antibody according to any of embodiments 1-50.

58. A method of treatment comprising administering an antibody according to any one of embodiments 1-50 to a subject.

59. A method for treatment of a metabolic disease or disorder comprising administering a therapeutically effective amount of an antibody according to any of embodiments 1-50 to a subject in need.

60. A method for treatment of overweight or obesity comprising administering a therapeutically effective amount of an antibody according to any of embodiments 1-50 to a subject in need.

61. A method for reducing body weight comprising administering a therapeutically effective amount of an antibody according to any one of embodiments 1-50 to a subject in need.

62. A method for treating type 2 diabetes, CVD or obesity comprising administering an antibody according to any one of embodiments 1-50 to a patient.

63. A method for treating overweight comprising administering an antibody according to any one of embodiments 1-50 to a patient.

64. A method for reducing weight comprising administering an antibody according to any one of embodiments 1-50 to a subject, such as a human subject.

65. The method of any of embodiments 5957-64, comprising administration of an effective dosage of the ALK7 antibody.

66. The method of any of embodiments 57-64, comprising administration of the ALK7 antibody to a human subject with a BMI above 27, such as a BMI above 28, 29, or 30.

67. A nucleotide sequence or pair of nucleotide sequences encoding an antibody according to any of embodiments 1-50.

68. A host cell expressing an antibody according to any of embodiments 1-50.

69. A method for producing an antibody according to any of embodiments 1-50.

70. A process for producing the antibody according to any of embodiments 1-50 comprising the steps of:
a) culturing a host cell expressing said antibody under condition conducive for expression of said antibody; and
b) purifying said antibody.

71. A pharmaceutical composition comprising an antibody according to any of embodiments 1-50.

72. The composition according to embodiment 71, wherein the composition is a liquid composition, such as an aqueous composition.

### Aspects

1. An anti-ALK7 antibody or antigen binding fragment thereof comprising:
(a) a heavy chain variable region comprising a hCDR1 of SEQ ID NO.: 62 or SEQ ID NO.: 63 with up to 1 amino acid substitution, a hCDR2 of any one of SEQ ID NOs.: 64-70 with up to 1 or 2 amino acid substitutions, and a hCDR3 of SEQ ID NO.: 71, or SEQ ID NO.: 72 with up to 1 or 2 amino acid substitutions, and
(b) a light chain variable region comprising a ICDR1 of any one of SEQ ID NOs.: 73-75 with up to 1 or 2 amino acid substitutions, ICDR2 of SEQ ID NO.: 76 or SEQ ID NO.: 77 with up to 1 amino acid substitutions, and ICDR3 of SEQ ID NO.: 78 or SEQ ID NO.: 79 with up to 1 amino acid substitution.
   2. The anti-ALK7 antibody or antigen binding fragment thereof of aspect 1 specifically binding to the extracellular domain of human ALK7, wherein the extracellular domain of human ALK7 comprises amino acid residues 26-113 of human ALK7 of SEQ ID NO.: 82.
   3. The anti-ALK7 antibody or antigen binding fragment thereof of aspect 1 or aspect 2, wherein the anti-ALK7 antibody or antigen binding fragment thereof comprises:
(a) a heavy chain variable region comprising a hCDR1 of SEQ ID NO.: 62 or SEQ ID NO.: 63, a hCDR2 of any one of SEQ ID NOs.: 64-70, and a hCDR3 of SEQ ID NO.: 71, or SEQ ID NO.: 72, and
(b) a light chain variable region comprising a ICDR1 of any one of SEQ ID NOs.: 73-75, ICDR2 of SEQ ID NO.: 76 or SEQ ID NO.: 77, and ICDR3 of SEQ ID NO.: 78 or SEQ ID NO.: 79.
   4. The anti-ALK7 antibody or antigen binding fragment thereof of any one of aspects 1-3, comprising:
(a) a heavy chain variable region comprising an amino acid sequence that is at least 90% identical to any one of SEQ ID NOs.: 1-34, and
(b) a light chain variable region comprising an amino acid sequence that is at least 90% identical to any one of SEQ ID NOs.: 35-59.
   5. The anti-ALK7 antibody or antigen binding fragment thereof of any one of aspects 1-4, comprising:
(a) a heavy chain variable region comprising an amino acid sequence of any one of SEQ ID NOs.: 1-34, and
(b) a light chain variable region comprising an amino acid sequence of any one of SEQ ID NOs.: 35-59.
   6. The anti-ALK7 antibody or antigen binding fragment thereof of any one of the previous aspects, wherein the anti-ALK7 antibody or antigen binding fragment thereof comprises:
(a) a heavy chain variable region comprising a hCDR1 of SEQ ID NO.: 63, a hCDR2 of SEQ ID NO.: 65, and a hCDR3 of SEQ ID NO.: 72, and
(b) a light chain variable region comprising a ICDR1 of SEQ ID NO.: 74, ICDR2 of SEQ ID NO.: 76, and ICDR3 of SEQ ID NO.: 78.
   7. The anti-ALK7 antibody or antigen binding fragment thereof of any one of aspects 1-6, comprising:
(a) a heavy chain variable region comprising an amino acid sequence of SEQ ID NO.: 18, and
(b) a light chain variable region comprising an amino acid sequence of SEQ ID NO.: 53.
   8. The anti-ALK7 antibody or antigen binding fragment thereof according to any one of the previous aspects, wherein anti-ALK7 antibody or antigen binding fragment thereof is a monoclonal antibody.
   9. The anti-ALK7 antibody or antigen binding fragment thereof according to any one of the previous aspects, wherein anti-ALK7 antibody or antigen binding fragment thereof is an IgG1 or IgG4 isotype.
   10. An anti-ALK7 antibody or antigen binding fragment thereof binding the extracellular domain of human ALK7 wherein the anti-ALK7 antibody or antigen binding fragment thereof specifically binds a non-contiguous ALK7 epitope that comprises amino acid residues 26-47 and 62-74 of human ALK7 of SEQ ID NO.: 82.
   11. The anti-ALK7 antibody or antigen binding fragment thereof according to any one of the previous aspects, wherein the anti-ALK7 antibody or antigen binding fragment thereof specifically binds a non-contiguous ALK7 epitope that comprises amino acid residues 26-47, 62-74, and 88-89 of human ALK7 of SEQ ID NO.: 82.
   12. The anti-ALK7 antibody or antigen binding fragment thereof according to any one of the previous aspects, wherein the anti-ALK7 antibody or antigen binding fragment thereof binds a non-contiguous ALK7 epitope which does not include amino acid residues 48-61 of human ALK7 of SEQ ID NO.: 82.
   13. The anti-ALK7 antibody or antigen binding fragment thereof according to any one of the previous aspects, wherein the anti-ALK7 antibody or antigen binding fragment thereof binds an ALK7 epitope that comprises at least eight amino acid residues selected from the group consisting of L26, C30, L32, C33, S35, S36, C40, Q41, T42, E43, G44, W47, S62, C63, V64, S65, L66, P67, E68, N70, A71, F74, and F88 relative to human ALK7 of SEQ ID NO: 82.
   14. The anti-ALK7 antibody or antigen binding fragment thereof according to any one of the previous aspect, wherein the anti-ALK7 antibody or antigen binding fragment thereof binds an ALK7 epitope comprising:
(i) optionally the amino acid residue L26 of human ALK7 of SEQ ID NO.: 82,
(ii) at least two amino acid residues selected from the group consisting of C30, L32, C33, S35, S36, C40, Q41, T42, E43, G44, and W47 of human ALK7 of SEQ ID NO.: 82,
(iii) at least two amino acid residues selected from the group consisting of S62, C63, V64, S65, L66, P67, E68, N70, A71, and F74 of human ALK7 of SEQ ID NO.: 82, and
(iv) optionally at least one amino acid residue selected from the group consisting of F88 and T89 of human ALK7 of SEQ ID NO.: 82.
   15. The anti-ALK7 antibody or antigen binding fragment thereof according to any one of aspects 10-14, wherein the ALK7 epitope is defined as amino acid residues within 5 Å of the antibody as determined by X-ray diffraction.
   16. The anti-ALK7 antibody or antigen binding fragment thereof according to any one of the previous aspects, wherein the anti-ALK7 antibody or antigen binding fragment thereof has an IC50 below 50 nM, such as below 10 nM.
   17. A pharmaceutical composition comprising an anti-ALK7 antibody or antigen binding fragment thereof according to any one of the previous aspects.
   18. An anti-ALK7 antibody or antigen binding fragment thereof of any one of aspects 1-16 or a composition according aspect 17 for use as a medicament.
   19. An anti-ALK7 antibody or antigen binding fragment thereof of any one of aspects 1-16, or a composition of aspect 17 for use in a method of treatment of a disease or disorder.

### Examples

Example 1: Generation of Antibodies from Hybridoma Cell Lines

### Immunization of ALK7-Knock Out (KO) Mice and Identification of Hits

Attempts to raise an immune response in wild type (wt) mice against human ALK7 failed, likely due to the very high identity between the extra-cellular domain of human and mouse ALK7. Therefore, constitutive ALK7 homozygous KO balb/c mice were generated by Taconic (Germantown, New York, US) by targeted deletion of exons 2-9 of the murine Acvrc1 gene. In order to generate high potency antibodies towards membrane localized and natively presented ALK7, the ALK7 KO mice were immunized with virus-like particles (VLP) comprising full length human ALK7 (Q8NER5-Uniprot identifier). The VLPs were produced by transient co-transfection of HEK293F cells with plasmids encoding (1) human ALK7 and (2) the gag-pol genes from HIV, using the Expi293 expression system (Thermo fisher scientific, US) kit. An immunization regime with one subcutaneous and two intraperitoneal immunizations with fourteen days between each immunization were employed using 20 μg VLP per mouse per immunization formulated in RIBI adjuvant (Sigma-Aldrich, US).

Ten days after the last immunization, the serum titer was analyzed by ELISA using an ALK7-Fc fusion construct (SEQ ID No.: 85) as antigen or by analyzing binding of serum to ALK7 transfected HEK293 cells using imaging.

Mice with positive titer were selected for intravenous (iv) boost with 5 μg of ALK7-VLPs in addition 5 μg of ALK7-HSA fusion protein (SEQ ID NO.: 84). Three days after boost, the spleen cells were harvested and fused to X63 myeloma cells using electroporation. The hybridoma cells were plated in 40 of 96 well plates and screened on ALK7 expressing HEK293 cells using a Celigo™ image cytometer (Revvity, Waltham, Massachusetts, US), using secondary goat anti-mouse IgG labelled with Alexa488 for detection.

Binding to ALK7 expressing HEK293 cells was evaluated using a Celigo algorithm calculating the ratio between the segmented area of fluorescent cells versus the segmented area of cells using brightfield (BF) imaging, to compensate for uneven distribution of cells in the assay plates.

By this procedure, 656 clones were provided, which bind to ALK7 expressing HEK cells (data not shown) The 656 clones were additionally screened for binding to ALK7 transfected C2C12 cells (ATCC, cat CRL-1772), see Table 1.1 below, as well as to non-transfected C2C12 cells (data not shown). In Table 1.1, a higher Celigo ratio value correlate with stronger binding of the antibodies to ALK7 transfected C2C12 cells.

A standard ELISA (Enzyme-Linked Immunosorbent Assay) assay was also performed on the 656 clones in order to demonstrate specific binding to an ALK7-HSA fusion protein (SEQ ID NO.: 84). A higher OD450 value indicates stronger binding of antibody to ALK7-HSA in Table 1.1.

Based on initial analysis 64 clones were selected. Results from ELISA and cell binding on C2C12 cells are provided in Table 1.1 below.

TABLE 1.1
ALK7 protein binding (ELISA) and ALK7 cellular
binding (imaging) of 64 antibodies

| | | Imaging, ALK7 expressing C2C12 cells. |
|---|---|---|
| Clone | ELISA, ALK7-HSA | Celigo ratio (segmented area |
| # | fusion (OD 450 nm) | fluorescence/segmented area Brigthfield) |
| 1. | 1.44 | 1.71 |
| 2. | 1.41 | 1.94 |
| 3. | 1.40 | 1.06 |
| 4. | 1.29 | 1.30 |
| 5. | 1.27 | 1.94 |
| 6. | 1.24 | 2.26 |
| 7. | 1.23 | 1.17 |
| 8. | 1.19 | 1.34 |
| 9. | 1.15 | 0.98 |
| 10. | 1.14 | 1.88 |
| 11. | 1.13 | 1.29 |
| 12. | 1.10 | 1.41 |
| 13. | 1.10 | 1.20 |
| 14. | 1.09 | 1.02 |
| 15. | 1.08 | 1.40 |
| 16. | 1.07 | 1.27 |
| 17. | 1.07 | 1.72 |
| 18. | 1.04 | 2.16 |
| 19. | 1.03 | 0.95 |
| 20. | 0.99 | 7.15 |
| 21. | 0.99 | 1.19 |
| 22. | 0.98 | 2.31 |
| 23. | 0.91 | 1.66 |
| 24. | 0.91 | 0.46 |
| 25. | 0.87 | 2.15 |
| 26. | 0.86 | 0.85 |
| 27. | 0.85 | 1.67 |
| 28. | 0.77 | 0.97 |
| 29. | 0.72 | 1.53 |
| 30. | 0.69 | 2.35 |
| 31. | 0.54 | 1.30 |
| 32. | 0.53 | 1.18 |
| 33. | 0.52 | 0.50 |
| 34. | 0.50 | 1.14 |
| 35. | 0.49 | 1.42 |
| 36. | 0.48 | 0.85 |
| 37. | 0.46 | 1.09 |
| 38. | 0.45 | 1.92 |
| 39. | 0.45 | 2.18 |
| 40. | 0.44 | 2.87 |
| 41. | 0.43 | 1.10 |
| 42. | 0.39 | 0.79 |
| 43. | 0.32 | 1.22 |
| 44. | 0.13 | 1.30 |
| 45. | 0.07 | 1.63 |
| 46. | 0.07 | 1.57 |
| 47. | 0.04 | 1.83 |
| 48. | 0.03 | 1.08 |
| 49. | 0.03 | 1.41 |
| 50. | 0.03 | 2.23 |
| 51. | 0.03 | 1.18 |
| 52. | 0.02 | 1.06 |
| 53. | 0.02 | 2.05 |
| 54. | 0.02 | 1.26 |
| 55. | 0.02 | 1.38 |
| 56. | 0.02 | 1.42 |
| 57. | 0.02 | 2.02 |
| 58. | 0.02 | 1.48 |
| 59. | 0.02 | 1.23 |
| 60. | 0.01 | 1.55 |
| 61. | 0.01 | 1.43 |
| 62. | 0.01 | 1.40 |
| 63. | 0.01 | 2.07 |
| 64. | 0.01 | 1.20 |

The hybridoma cells expressing the 64 positive binders (ELISA positive, imaging positive or both ELISA and imaging positive) from Table 1.1 were expanded and their secreted antibodies affinity purified on protein A. The antibodies were further tested for functional inhibition of Activin B induced activation of ALK7 transfected HEK293 cells (RGA assay, Example 4) and functional inhibition of smad2 and smad3 phosphorylation in human adipocytes (p-SMAD3 assay, Example 4). Out of the 64 initial hybridoma clones binding to ALK7 transfected C2C12 cells, only three mAbs displayed significant functional inhibition of Activin B induced ALK7 activation, i.e. antibody clones #4, 10 and 19. These three antibodies were sequenced and recombinantly expressed.

The hybridoma clone #19 resulted in ambiguous DNA sequence data and were cloned in two different Expi293F light chain variable region constructs (19a and 19b).

The three clones were recombinantly expressed as human IgG1m3 allotype IgG comprising engineered silencing mutations (L117A, L118E, G120A, A213S and P214S) (SEQ ID NO.: 60 and 61) in order to abolish Fc receptor and complement system interactions Transfection was performed by manufacturer's protocol of Expi293F transient transfection. After 5 days of cultivation, cultures were harvested and filtered using 0.22 μm PES filter system. The binding and functional inhibition of ALK7 was confirmed and results included in Table 1.2 below.

TABLE 1.2
Functional characterization of clone # 4, # 19
and # 10 and their recombinant versions in SPR,
RGA and pSMAD3 assays. SPR was performed according to
details in Example 3, RGA and pSMAD3 assay according to Example 4.

| | | | | IC50, | |
|---|---|---|---|---|---|
| | | | | AlphaLISA | |
| | | | | SureFire, | |
| | | | IC50 - RGA | Ultra | SPR, |
| | | | ALK7 | p-SMAD3 | KD |
| Source | clone# | mAb | SBE-Luc(nM) | (nM) | (nM) |
| hybridoma | 4 | NA | 2.0 (n = 4) | 2.3 (n = 6) | ND |
| hybridoma | 19 | NA | 1.0 (n = 34) | 0.52 (n = 15) | 47 |
| | | | | | (n = 4) |
| hybridoma | 10 | NA | 3.2 (n = 5) | 2.6 (n = 3) | ND |
| Recombinant | 4 | A | 4.4 (n = 2) | 2.2 (n = 2) | ND |
| Recombinant | 19a | B1 | 1.1 (n = 12) | 0.46 (n = 2) | 35 |
| | | | | | (n = 4) |
| Recombinant | 19b | B2 | 46.0 (n = 1) | ND | ND |
| Recombinant | 10 | NA | 1.5 (n = 4) | 2.1 (n = 1) | ND |
| Recombinant | 10 | NA | 4 (n = 3) | ND | ND |

### Example 2: Humanization of Mouse Antibody Clone 4 and 19a

The selected clones from the functional inhibition assay were humanized (Harding et al., MAbs 2010; 2:256-265) through a series of design cycles, first, applying the large language models Sapiens (Prihoda et al., mAbs 2022, 14, e2020203) and MetaAI ESM (Lin et al., Science 2023, 379, 1123-1130) in combination with traditional CDR grafting to a human IgG1 format extending the Sapiens amino acid space with a Gibbs sampling of amino acid combinations using a random-walk approach and a down-sampling in the ESM embedding space for maximum diversity, within natural human antibody repertoire sequence space, to allow a larger sequence variation in the framework regions of the variable domain than what would be achieved by traditional CDR grafting using Kabat CDR definitions on nearest human germ line and also allowing a broader mapping of the amino acid positions important for functional and developability properties. Introduction of Gly and Cys amino acid residues were not allowed by deselection. The antibodies from this sequence expansion was further optimised towards the Sapiens amino acid space and acceptable functional properties as well as developability and processability using read-outs from biophysical evaluation (Example 6 and Estep et al., 2015, mAbs, 7, 553-561)), SPR (Example 3) and potency (RGA, Example 4) in-vitro assays, sequence based chemical liability predictors from Bio MOE (Molecular Operating Environment (MOE) Montreal (QC, Canada): Chemical Computing Group ULC), expression yields, predicted MHCII binding (Reynisson et al. Nucleic Acids Res, W48, W449-W454, 2020) and the crystal structure of the target complex Fab A (Example 7) as guidance.

The following antibody and antibody variants were produced and characterized as described further below. The antibodies included the same heavy and light chain constant regions identified in the sequence listing by SEQ ID NO.: 60 and 61, respectively, except for V45 and V46, which includes three additional amino acid changes (E155C, R175C and V185C) compared to IgG1m3 in the heavy chain constant region.

TABLE 2.1
Table overview of the heavy and light chain variable
region sequences of the tested antibodies.

| | Heavy Chain | Light Chain |
|---|---|---|
| | Variable region | Variable region |
| mAb | (SEQ ID No.:) | (SEQ ID No.:) |
| A | 1 | 35 |
| B1 | 2 | 36 |
| B2 | 2 | 37 |
| V1 | 3 | 38 |
| V2 | 4 | 39 |
| V3 | 4 | 40 |
| V4 | 5 | 41 |
| V5 | 6 | 41 |
| V6 | 7 | 42 |
| V7 | 8 | 43 |
| V8 | 9 | 44 |
| V9 | 9 | 45 |
| V10 | 10 | 39 |
| V11 | 11 | 39 |
| V12 | 12 | 46 |
| V13 | 13 | 47 |
| V14 | 13 | 48 |
| V15 | 13 | 49 |
| V16 | 13 | 50 |
| V17 | 14 | 50 |
| V18 | 15 | 50 |
| V19 | 16 | 51 |
| V20 | 17 | 51 |
| V21 | 18 | 47 |
| V22 | 19 | 52 |
| V23 | 18 | 53 |
| V24 | 8 | 54 |
| V25 | 8 | 55 |
| V26 | 20 | 55 |
| V27 | 8 | 56 |
| V28 | 21 | 52 |
| V29 | 22 | 52 |
| V30 | 23 | 57 |
| V31 | 22 | 57 |
| V32 | 24 | 57 |
| V33 | 25 | 57 |
| V34 | 26 | 57 |
| V35 | 27 | 53 |
| V36 | 26 | 53 |
| V37 | 28 | 58 |
| V38 | 22 | 58 |
| V39 | 25 | 58 |
| V40 | 29 | 59 |
| V41 | 30 | 59 |
| V42 | 23 | 59 |
| V43 | 20 | 59 |
| V44 | 25 | 59 |
| V45 | 18 | 53 |
| V46 | 26 | 53 |
| V47 | 31 | 53 |
| V48 | 32 | 53 |
| V49 | 33 | 47 |
| V50 | 34 | 57 |

The antibodies characterized comprise a selection of different CDR's as identified according to kabat as included in Table 2.2.

TABLE 2.2
CDR sequences as identified by Kabat
from the different mAb variants.

| | SEQ | | SEQ |
|---|---|---|---|
| Heavy chain | ID NO: | Light chain | ID NO: |
| CDRH1 | | CDRL1 | |
| SGNYWN | 62 | KSSQSLLNSRTRKNYLA | 73 |
| SGSYWN | 63 | KSSQSLLVSRTRKNYLA | 74 |
| | | KSSQSLLYSRTRKNYLA | 75 |
| CDRH2 | | | |
| YISFDGRNNYNPSLKN | 64 | CDRL2 | |
| YISFDGRTNYNPSLKN | 65 | WTSTRES | 76 |
| YISFDGRTNYNPSLKS | 66 | WASTRES | 77 |
| YISFDGRRNYNPSLKN | 67 | | |
| YISFTGRTNYNPSLKN | 68 | CDRL3 | |
| YISFSGRNNYNPSLKN | 69 | KQSYNLPYT | 78 |
| YISFSGRTNYNPSLKN | 70 | KRSYNLPYT | 79 |
| CDRH3 | | | |
| DYYGSEGFGY | 71 | | |
| DYYGSEGFAY | 72 | | |

As seen a series of variants was created including few amino acids changes in the CDR sequence as illustrated by the comparison of A with V23 and V36 in Tables 2.3 and 2.4 below.

TABLE 2.3
Table overview of selected heavy chain
CDR sequences (Kabat) for different antibodies.

| | CDRH1 | CDRH2 | CDRH3 |
|---|---|---|---|
| A | SGNYWN | YISFDGRNNYNPSLKN | DYYGSEGFGY |
| | (SEQ ID | (SEQ ID NO.: 64) | (SEQ ID |
| | NO.: 62) | | NO.: 71) |
| V23 and | SGSYWN | YISFDGRTNYNPSLKN | DYYGSEGFAY |
| V36 | (SEQ ID | (SEQ ID NO.: 65) | (SEQ ID |
| | NO.: 63) | | NO.: 72) |

TABLE 2.4
Table overview of selected light CDR sequences
(Kabat) for different antibodies indicating
few amino acid changes in V23/V36 compared to A.

| | CDRL1 | CDRL2 | CDRL3 |
|---|---|---|---|
| A | KSSQSLLNSRTRKNYLA | WTSTRES | KQSYNLPYT |
| | (SEQ ID NO.: 73) | (SEQ ID | (SEQ ID |
| | | NO.: 76) | NO.: 78) |
| V23 and | KSSQSLLVSRTRKNYLA | WTSTRES | KQSYNLPYT |
| V36 | (SEQ ID NO.: 74) | (SEQ ID | (SEQ ID |
| | | NO.: 76) | NO.: 78) |

### Example 3: Binding Affinity to the Extracellular Domain (ECD) of ALK7 (ALK7-ECD) Using SPR

Surface Plasmon Resonance (SPR) binding studies were performed using either Biacore™ T200 (Cytiva AB, Uppsala), Biacore™ 8K (Cytiva AB, Uppsala), Biacore™ 8K+ (Cytiva AB, Uppsala), Biacore™ 1S+ (Cytiva AB, Uppsala) or Sierra SPR®-32 Pro (Bruker Daltronics) instruments. The experiments were conducted at 25° C., and samples were maintained at 10-25° C. in the sample compartment chamber. In the assay, ALK7-ECD was provided as a fusion protein of ALK7 aa 26-113 and HSA (SEQ ID NO.: 80).

For testing the generated ALK7 antibodies we used a sensor chip (Protein A/G sensor chip (XanTec bioanalytics GmbH, Duesseldorf), Protein A sensor chip (Cytiva AB, Uppsala) or IgG Capture sensor chip (Bruker Daltronics)). The chip surface was prepared by rehydration in HBS-EP (0.01 M HEPES pH 7.4, 0.15 M NaCl, 3 mM EDTA, 0.005% v/v Surfactant P20), buffer in the instrument using stand-by flow. Prior to the experiment the chip surface was conditioned using Regeneration solution (10 mM Glycine-HCl pH 1.5, Cytiva; BR 100354) followed by three consecutive start-up cycles using buffer as analyte. Each cycle consisted of the following steps: 1) Ligand capture using ALK7 antibodies, at 1.5 to 15 μg/mL (Flow rate: 10 μL/min, Contact time: 20-60 seconds) 2) ALK7-ECD Analyte injection (Flow rate: 30-50 μL/min, Contact time: 240-400 seconds, Dissociation time: 400-1200 seconds) and 3) Regeneration using Regeneration solution (Flow rate 30 μL/min Contact time 30 sec). The ALK7-ECD analyte samples were serial diluted in HBS-EP buffer before being applied to the antibody coated chip.

Resulting binding responses were subject to subtraction of reference surface signals as well as blank buffer injections over captured antibodies. This process allowed for the correction of instrument noise, bulk shift, and drift during sample injections. The association and dissociation rate constants, namely kₐ (association rate) and k_{d} (dissociation rate), were extracted by globally fitting a 1:1 Langmuir model to the data using an appropriate software (Biacore T200 Evalutation Software (Cytiva AB, Uppsala), Biacore Insight Evaluation Software (Cytiva AB, Uppsala) or SPR Analyzer (Bruker Daltronics)). The intrinsic affinities between the various antibodies and the ALK7-ECD-HSA were reported as the equilibrium dissociation constant (KD), determined as the relationship between the reaction rates (K_{D}=K_{d}/kₐ). We also refer to these values as the ‘intrinsic affinities’. Values are included in Table 4.1 below.

### Example 4: Functional Assays and Characterisation of ALK7 Antibodies

### Receptor Potency Using RGA Assay

A reporter gene assay (RGA) was established to determine the ability of anti-ALK7 antibodies to inhibit ALK7 signaling. The luciferase gene was used, under control of Smad binding element (SBE) to measure ALK7 signaling, since stimulated ALK7 is known to phosphorylate Smad3, which binds SBE. Thus, stimulation of ALK7 in cells containing SBE-luciferase results in increased luciferase expression.

HEK293 cells (ATCC CRL-1573) were stably transfected with the SBE-luciferase reporter plasmid pGL4.48[luc2P/SBE/Hygro] from Promega and a single cell clone was isolated. This single cell clone was stably transfected with a human ALK7-S270T expression plasmid based on pcDNA3.1 (+) and a single cell clone, named clone 6, was isolated. The S270T mutation renders ALK7 insensitive to the SB431542 inhibitor, which was subsequently used to avoid stimulation of endogenous ALK receptors.

For measuring inhibition of ALK7 signaling, the HEK293/hALK7-S270T/SBE-luciferase (clone 6) cells were seeded in white, opaque 384-well plates at a density of 20000 cells/well. The following day medium was removed from the assay plates and compounds diluted in medium containing 1.36 μM SB431542 was added. After 30 min incubation at 37° C., 0.5 nM human Activin B (R&D Systems) diluted in medium containing 1.36 μM SB431542 was added followed by a 4 h incubation at 37° C. Cells were lysed and luciferase substrate added using the Steady-GLO kit (Promega). Luminescence was detected using the EnVision 2104 plate reader with ultrasensitive luminescence detection (PerkinElmer). DMEM supplemented with 10% fetal bovine serum and 1% penicillin/streptomycin was used for seeding cells and dilution of compounds and agonist.

For determining IC₅₀ values, antibody concentration-response curves were fitted to the four-parameter logistic equation. The results obtained are included in the below Table 4.1.

TABLE 4.1
Functional characterization of antibodies
as determined by SPR and the RGA assay.

| | Affinity/SPR data | ALK 7 inhibition/RGA assay |
|---|---|---|
| Mab # | K_{D} (nM) | IC50 (nM) |
| A | 35 | 1.4 |
| B1 | No Data | 4.4 |
| V1 | 813 | 1.1 |
| V2 | 52 | 1.5 |
| V3 | 46 | 0.8 |
| V4 | 505 | 0.5 |
| V5 | 316 | 1.1 |
| V6 | 68 | 0.7 |
| V7 | 318 | 2.8 |
| V8 | 186 | 2.3 |
| V9 | 329 | 2.4 |
| V10 | 50 | 1.2 |
| V11 | 50 | 0.8 |
| V12 | 794 | 3.9 |
| V13 | 60 | 1.8 |
| V14 | 17 | 2.3 |
| V15 | 118 | 1.2 |
| V16 | 31 | 2.8 |
| V17 | 17 | 1.4 |
| V18 | 26 | 2.3 |
| V19 | 24 | 2.5 |
| V20 | 33 | 2.5 |
| V21 | No Data | 2.0 |
| V22 | 210 | 2.1 |
| V23 | 55 | 1.6 |
| V24 | 142 | 1.5 |
| V25 | 21 | 1.5 |
| V26 | 110 | 1.4 |
| V27 | 25 | 2.1 |
| V28 | 132 | 1.8 |
| V29 | 189 | 1.8 |
| V30 | 16 | 1.5 |
| V31 | 20 | 2.1 |
| V32 | 11 | 1.5 |
| V33 | 16 | 2.0 |
| V34 | 17 | 1.1 |
| V35 | 102 | 1.6 |
| V36 | 130 | 1.7 |
| V37 | 154 | 1.7 |
| V38 | 21 | 1.5 |
| V39 | 14 | 1.3 |
| V40 | 82 | 1.1 |
| V41 | 94 | 1.3 |
| V42 | 12 | 1.7 |
| V43 | 13 | 1.7 |
| V44 | 16 | 1.6 |
| V45 | 121 | No data |
| V46 | 117 | No data |
| V47 | 188 | 1.6 |
| V48 | 41 | 1.6 |
| V49 | 34 | 1.4 |
| V50 | 22 | 1.5 |

The results obtained in the SPR and RGA assays shows that the identified antibodies display a moderate to high binding affinity and that the antibodies are potent ALK7 inhibitors. We found the inhibitor potency to be markedly increased compared to the measured affinity from SPR indicative of avidity arising from bivalent interactions. I.e. mAb variants have a KD in the range of 10-850 nM and potencies determined in the RGA assay of 0.8-4.4 nM. i.e. mAb variants V2, V7, V8, V22 and V29 have quite different K_{D}'s (in the range of 192-343 nM) while their potencies determined in the RGA assay are increased two orders of magnitude (1.6-2.8 nM).

p-SMAD3 Signalling in Human Adipocyte Cells

### Primary Human Subcutaneous Preadipocytes

Primary Human Subcutaneous Preadipocytes are isolated from subcutaneous adipose tissue by enzymatic digestion and selective culturing techniques and purchased frozen as unpassaged preadipocytes in single use cryotubes. Once thawed, cells are suspended in Preadipocyte Growth Medium-2 supplemented with Fetal bovine serum, L-glutamine, and GA-1000 SingleQuots™, and 10.000 cells/well were dispensed in 96 well plates and incubated at 37° C., 5% CO₂ and 90% humidity for 24 hours. Preadipocytes are started to be differentiated the next day by adding Growth Medium-2 containing above supplements with addition of insulin, dexamethasone, indomethacin and isobutyl-methylxanthine and let to differentiate for 10 days. After differentiation cells are starved for 24 hours in subcutaneous basal medium containing bovine serum albumin and assayed the next day.

p-SMAD3 Assay

Assay kit used was the AlphaLISA® SureFire® Ultra™ p-SMAD3 (Ser423/Ser425) assay which is a sandwich immunoassay for quantitative detection of phospho-SMAD3 (phosphorylated on Ser423/Ser425) in cellular lysates using Alpha Technology. Starving media was removed from the cells and recombinant human Activin B (an ALK7 receptor ligand), at a constant concentration, and ALK7 blocking antibodies, serial diluted, in subcutaneous basal medium containing tween was added to the cells and let to incubate for 40 min. Media was then removed and lysis buffer was added to the cells and plates were flash frozen to ensure proper cell lysis. 10 μl of cell lysate was transferred to untreated AlphaPlate-384 and 5 μl of acceptor bead mix was added to wells and incubated for 1 hour followed by addition of 5 μl of donor bead mix and incubated over night at room temperature protected from light. Plates were read on an EnVision™ plate reader (excitation 680 nm, emission 615 nm) and IC₅₀ values were calculated in Graph Pad Prism by non-linear regression curve fitting.

Example 5: Simulation of Avidities from Monovalent Rate Constants

The bivalency of antibodies allows one antibody molecule to bind to two antigen sites. This may significantly increase the rebinding probability and thus decrease the macroscopic dissociation rate constant of the antibody: antigen complex. This is often referred to as the ‘functional affinity’ or ‘avidity’. Depending on the experimental design, SPR can be used to measure the intrinsic affinity, that is the affinity between one antibody arm and the one antigen. The functional assay such as the RGA potency assay presented in Example 4, relies on cellular expression of the antigen which may, in cases where both antibody arms are capable of binding two antigens expressed on the cell surface, report avidities several orders of magnitude higher than the intrinsic affinities measured by SPR. Therefore, as demonstrated in Example 4, antibodies may have a high potency relevant for a therapeutic in vivo application in the RGA assay despite a modest affinity in SPR.

To determine whether the potencies measured in the RGA assay in Example 4 are avidities as a result of the valency, we used the intrinsic affinities and underlying rate constants to simulate the avidities. The avidities were simulated by deterministically solving sets of differential equations as a function of time and at various antibody concentrations, [ab]. The binding of the first antibody arm to the first receptor, abAB or ABab complex, will usually be closely related to the intrinsic affinity which can be measure by SPR. However, in cases where a second receptor is within reach of the second arm, the resulting avidity (the aABb complex) can be approximated by calculating a local concentration, L, related to the distance between the two antibody arms, r, which will greatly enhance the association rate of the second binding event. L is defined as:

$$L = \frac{( \frac{1}{{N}_{A}} )}{( ( \frac{2}{3} ) * π * {r}^{3} * 1000 )} , where {N}_{A} is Avogadro ’ s number$$

L can then be used in the set of differential equations displayed below. Additional parameters are; k1: Association rate constant of the first arm in (1/Ms), k2: Dissociation rate constant of the first arm (1/s), k3: Association rate constant of the second arm (1/Ms), k4: Dissociation rate constant of the second arm (1/s), f: The penalty factor (unitless and set to 1 in this case). The initial concentration of ALK7 receptors, [AB], is preset to 1E-10 M. In this case, k1=k3 and k2=k4 and are determined from SPR experiments. The avidity is estimated at 50% AB occupancy. Simulations are allowed to equilibrate for timings similar to those used in the RGA functional assay. Differential equations are written out below.

$$\frac{d ( [ A B ] )}{d t} = - k 1 * [ ab ] * [ A B ] + k 2 * [ a b A B ] - k 3 * [ ab ] * [ A B ] + k 4 * [ ABab ]$$

$$\frac{d ( [ a b ] )}{d t} = - k 1 * [ ab ] * [ A B ] + k 2 * [ a b A B ] - k 3 * [ ab ] * [ A B ] + k 4 * [ A B a b ] - k 3 * [ a b A B ] * [ a b ] + k 4 * [ a b A B a b ] - k 1 * [ ABab ] * [ a b ] + k 2 * [ abABab ]$$

$$\frac{d ( [ a b A B ] )}{d t} = + k 1 * [ ab ] * [ AB ] - k 2 * [ a b A B ] - ( \frac{L}{f} ) * k 3 * [ a b A B ] + k 4 * [ a A B b ] - k 3 * [ a b A B ] * [ a b ] + k 4 * [ abABab ]$$

$$\frac{d ( [ A B a b ] )}{d t} = + k 3 * [ ab ] * [ A B ] - k 4 * [ A B a b ] - ( \frac{L}{f} ) * k 1 * [ A B a b ] + k 2 * [ a A B b ] - k 1 * [ ABab ] * [ ab ] + k 2 * [ abABab ]$$

$$\frac{d ( [ a A B b ] )}{d t} = + ( \frac{L}{f} ) * k 3 * [ a b A B ] - k 4 * [ a A B b ] + ( \frac{L}{f} ) * k 1 * [ A B a b ] - k 2 * [ a A B b ]$$

$$\frac{d ( [ a b A B a b ] )}{d t} = + k 3 * [ a b A B ] * [ a b ] - k 4 * [ a b A B a b ] + k 1 * [ ABab ] * [ a b ] - k 2 * [ abABab ]$$

The table below compares the affinities obtained from SPR (Example 3), the simulated avidities and the potencies from the RGA assay (Example 4). A good correlation between the measured RGA potencies and the simulated avidities was observed.

TABLE 4.2
Simulated avidities. The measured potencies (Table 4.1) are
compared to both the theoretical combined affinity (without
avidity contribution) and the simulated avidities.

| | | | Simulated Avidity |
|---|---|---|---|
| | Simulated | SPR Affinity vs. | vs. |
| | Avidity | Measured Potency | Measured Potency |
| Mab # | KD app (nM) | Fold (log10) | Fold (log10) |
| A | 0.6 | 64 | 0.4 |
| V1 | 1.3 | 623 | −0.1 |
| V2 | 0.6 | 93 | 0.4 |
| V3 | 0.3 | 159 | 0.4 |
| V4 | 1.5 | 333 | −0.5 |
| V5 | 0.3 | 941 | 0.5 |
| V6 | 0.3 | 209 | 0.3 |
| V7 | 0.5 | 667 | 0.8 |
| V8 | 0.7 | 279 | 0.5 |
| V9 | 0.8 | 402 | 0.5 |
| V10 | 0.8 | 62 | 0.2 |
| V11 | 0.4 | 132 | 0.3 |
| V12 | 1.0 | 798 | 0.6 |
| V13 | 1.1 | 55 | 0.2 |
| V14 | 1.4 | 12 | 0.2 |
| V15 | 1.9 | 63 | −0.2 |
| V16 | 1.8 | 17 | 0.2 |
| V17 | 1.6 | 11 | −0.1 |
| V18 | 1.6 | 16 | 0.2 |
| V19 | 1.1 | 22 | 0.4 |
| V20 | 1.1 | 30 | 0.4 |
| V21 | NA | NA | NA |
| V22 | 0.6 | 368 | 0.6 |
| V23 | 0.9 | 60 | 0.2 |
| V24 | 1.6 | 88 | 0.0 |
| V25 | 1.3 | 16 | 0.1 |
| V26 | 1.5 | 73 | 0.0 |
| V27 | 1.1 | 23 | 0.3 |
| V28 | 2.2 | 59 | −0.1 |
| V29 | 3.1 | 61 | −0.2 |
| V30 | 1.9 | 8 | −0.1 |
| V32 | 1.0 | 11 | 0.2 |
| V33 | 1.3 | 12 | 0.2 |
| V34 | 1.2 | 14 | 0.0 |
| V35 | 2.0 | 50 | −0.1 |
| V36 | 2.3 | 57 | −0.1 |
| V37 | 1.7 | 88 | 0.0 |
| V38 | 1.2 | 18 | 0.1 |
| V39 | 1.2 | 12 | 0.0 |
| V40 | 1.6 | 51 | −0.2 |
| V41 | 1.7 | 55 | −0.1 |
| V42 | 1.2 | 10 | 0.2 |
| V43 | 1.1 | 12 | 0.2 |
| V44 | 1.3 | 12 | 0.1 |
| V45 | 2.2 | 55 | NA |
| V46 | 2.2 | 55 | NA |
| V47 | 0.5 | 352 | 0.5 |
| V48 | 0.8 | 53 | 0.3 |
| V49 | 0.7 | 52 | 0.3 |
| V50 | 1.7 | 13 | −0.1 |

Differences are provided in log10 fold.

### Example 5A: In Vivo PK/PD Study in DIO Mice

Diet induced obese (DIO) mice were fed high fat diet (HFD) from beginning at 10 weeks of age. Mice were fed ad libitum HFD, until the cohort reached an average body weight>50 g. They were then dosed once with a subcutaneous injection of either 10 or 20 mg/kg body weight of an anti-ALK7 antibody in an aqueous formulation. Body weight and food intake was measured daily relative to a placebo group. The results are provided in Table 5.1.

TABLE 5.1
Half-life and weight loss relative to
placebo group after one injection.

| | Single Dose | | Weight Change | Terminal Weight |
|---|---|---|---|---|
| Mab | (SC) mg/kg | Half-life (h) | at day 21 | Change at day 31 |
| V7 | 20 | 130 | −13% | −9% |
| V13 | 10 | 83 | −11% | −5% |
| V13 | 20 | 56 | −12% | −4% |
| V25 | 10 | 67 | −12% | −10% |
| V23 | 10 | 68 | −10% | −11% |
| V36 | 10 | 236 | −7% | −11% |

ND not determined.

The results in Table 5.1 demonstrate that a single dose of the antibodies results in a significant weight loss in DIO mice over an extended period of time. This further shows that the antibodies have a potential as a therapeutic for use in treatment of metabolic diseases and further confirms that antibodies are suitable for infrequent dosing which is attractive for patients.

### Example 5B: Multiple Dose Study of V13

To study the weight loss and the change of fat and lean mass, respectively, over time DIO mice were administered multiple doses of compound V13.

Diet induced obese (DIO) mice were fed 45% high fat diet (HFD) from Research Diets (cat #D12451) beginning at 10 weeks of age. Mice we fed ad libitum HFD. From 40 weeks of age, with an average body weight>50 g, DIO mice received weekly (QW) subcutaneous (SC) injections of vehicle control or V13 mAb at 1, 5, or 10 mg/kg for a period of 28 days (i.e. 4 doses in total). Body weight and food intake was measured daily. Body composition was measured at day 28 using magnetic resonance imaging (MRI).

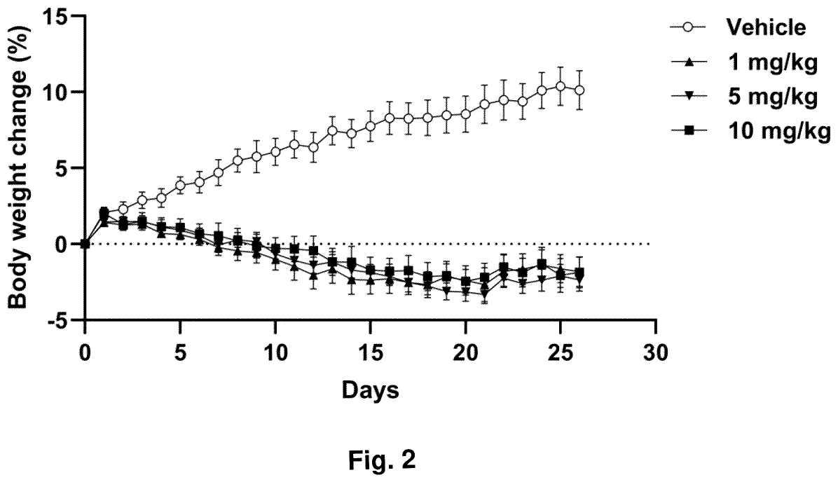

The percent weight change from baseline was calculated and is shown in FIG. 2. The mice receiving the control vehicle gained around 10% weight over the time of the study, while the mice receiving the anti-ALK7 antibody reduced body weight.

Mice given V13 lost on average around 18% fat mass and gain an average of around 7% lean mass at day 28.

TABLE 5.2
The changes in fat mass, lean mass and body
weight at day 28 after 4 doses of V13.

| | Fat Mass (%) | Lean Mass (%) | Body weight |
|---|---|---|---|
| Veicle | +12.7 | +2.7 | +10.1 |
| 1 mg/kg | −16.9 | +6.4 | −2.4 |
| 5 mg/kg | −17.3 | +7.8 | −1.8 |
| 10 mg/kg | −18.5 | +7.4 | −2.0 |

### Example 6: Biophysical Characterization—Viscosity and Solubility

Therapeutic antibodies often need to be formulated at a concentration above 100 mg/mL to make sub-cutaneious administration possible. The antibody should be soluble at this high concentration and preferably have a low viscosity in the formulation to enable the use of a sub-cutaneious injection device with a small needle gage.

Antibody samples were concentrated and buffer-exchanged to ≥150 mg/mL in 20 mM HEPES, 150 mM NaCl (pH 7.4) using an Amicon centrifugal concentrator (MWCO: 30 kDa). Concentrations were measured by absorbance at 280 nm using the Lunatic UV/Vis spectrophotometer (Unchained Labs). Following concentration, viscosity measurements were carried out using the VROC Initium instrument (Rheosense). 50 μL of each sample were loaded into HPLC vials with caps (Part No.: 186000384C, Waters). Vials were placed in the autosampler of the instrument, whereafter viscosity was measured over a shear rate range of 1000-4000 s⁻¹ at 5 and 20° C. Data was collected using the software of the Rheosense instrument and the results included in table 6.1 below.

TABLE 6.1
Viscosity as measured at 20° C., 2000 s⁻¹.

| | | | Viscosity |
|---|---|---|---|
| | Concentration | Solubility | (20° C., 2000 s⁻¹) |
| mAb | (mg/mL) | (visual inspection) | (cP) |
| A | 20* | Precipitated | N/A |
| V11 | 150 | Clear | 21 |
| V13 | 156 | Clear | 12 |
| V36 | 155 | Clear | 11 |
| V23 | 153 | Clear | 13 |

*Concentration measured in soluble fraction of precipitated sample during concentration step.

The antibodies V11, V13, V36, and V23 are soluble at 150 mg/mL and V13, V36, and V23 also demonstrate a low viscosity. The solubility and viscosity results in Table 6.1 shows that mAb V13, V36 and V23, by being soluble and having relatively low viscosities at high concentration, have improved properties with regard to syringe-ability and processability.

### Example 7: Epitope Characterization for Selected Antibodies

The epitope and paratopes of two mAb's where characterised by X-ray crystallography of their corresponding Fab fragments in complex with the extracellular domain of ALK7.

### Example 7a: Epitope Mapping of the Anti-ALK7 Fab of mAb a (Fab A), Using X-Ray Crystallography.

ALK7-ECD₂₆₋₁₁₃ (SEQ ID NO.: 83) for X-ray crystallography was produced in Expi293F GnT1 cells with a His-tag and purified using Ni-affinity chromatography (IMAC) using a 5 mL His Trap excel column in 20 mM HEPES, 300 mM NaCl, pH 7.4 buffer and then eluting using the same buffer with added 500 mM imidazole adjusted to pH 7.4 with HCl. The IMAC purified sample was deglycosylated using Endo Hf (P0703L, New England Biolabs) using approximately 4 μL Endo Hf per 1 mg target protein, followed by overnight cleavage at 30° C. The digested protein was then purified by size-exclusion chromatography on a HiLoad 26/60 Superdex75 column with a running buffer of 20 mM HEPES, 150 mM NaCl PH 7.4. Finally, product was concentrated on an Amicon Ultra 15 device with a 3 kDa cutoff. Product quality was verified by SE-HPLC, SDS-PAGE and LC-MS.

Fabs of mAb A and V12 for X-ray crystallography were produced in Expi cells and purified from cell free supernatant using a 5 mL HiTrap ProteinL column and a 20 mM formic acid, 8 mM NaOH, pH 3.5 elution buffer, followed by pH neutralization by addition of 500 mM Na2HPO4 in a 3:1 volume ratio. The material was further purified using a CaptureSelect CH1 XL affinity chromatography step. Protein was loaded at pH 7 and eluted using a 50 mM acetic acid, 75 mM NaCl, 8.35 mM NaOH, pH 3.5 elution buffer, followed by pH neutralization with a 1 M HEPES, 0.5 M NaOH, pH 8.0 solution added to the eluate in a 3:1 volume ratio. The eluate was finally buffer exchanged to 20 mM HEPES, 150 mM NaCl, pH 7.4 on a HiPrep 26/10 column. The Fabs A and V12, included the variable sequences of mAb A and V12 as described above and the heavy and light chain constant regions defined by SEQ ID NO.: 80 and 81 including a His tag on the heavy chain constant region.

### Crystallisation

The Fab fragment of mAb A was mixed in a 1:2 molar ratio with ALK7 ECD. The complex was subjected to size exclusion chromatography on a HiLoad 16/60 Superdex 75 pg column (GE Healthcare) run with 20 mM HEPES, pH 7.5, 140 mM NaCl buffer. The fractions containing the Fab/ALK7 ECD complex were pooled and concentrated to 9.9 mg/ml. Prior to crystallization, a VHH developed to bind the constant part of the Fab light chain, as described in reference PNAS 118 (47), e2115435118, 2021, was added in a 1:1 ratio. Crystals of the Fab A/ALK7 ECD/VHH complex were grown using sitting drop vapour diffusion at 20° C. The crystal used was grown using a protein solution of 150 nL 8.5 mg/ml complex in 20 mM Hepes, pH 7.4, 140 mM NaCl mixed with 150 nL of 0.1 M DL-Malic acid, MES monohydrate, Tris, pH 4.0 buffer, 25% (w/v) PEG 1500 as precipitant and incubated over 80 μL precipitant.

### Diffraction Data Collection

The crystal was cryo protected in precipitant added 20% of ethylene glycol prior to flash cooling in liquid nitrogen. Diffraction data were collected at 100K at the Swiss Light Source beamline X10SA (1.0000 Å wavelength) using an Eiger2 16M pixel detector from Dectris. Autoindexing, integration and scaling of the data were performed with programmes from the XDS package (diffracting data statistics are summarised in Table 7.1).

### Crystal Structure Determination and Refinement

The asymmetric unit contains one Fab A/ALK7 ECD/Fab-binding V_{H}H complex. The structure was determined by molecular replacement using Phaser as implemented in the programme suite Phenix using a Fab structure model of mAb A created by AlphaFold2 as search model. The V_{H}H was introduced from chain K of PDB entry 7PIJ after superposition chain L from this PDB entry on the light chain of Fab A. Finally, an AlphaFold2 model of ALK7 ECD was manually positioned in the difference density map. The full structure model was refined using steps of Phenix refinement and manual rebuilding in COOT. The refinement statistics are found in Table 7.1.

TABLE 7.1
Data collection and refinement statistics for Fab A/ALK7
ECD/Fab-binding VHH complex crystal structure.

| Wavelength (Å) | 1.0000 | |
|---|---|---|
| Resolution range (Å) | 45.88-3.25 | (3.45-3.25)* |
| Space group | P64 | |
| Unit cell (Å, deg) | 131.9 131.9 100.2 90 90 120 | |
| Total reflections | 165142 | (28381) |
| Unique reflections | 30494 | (5107) |
| Multiplicity | 5.4 | (5.6) |
| Completeness (%) | 99.47 | (99.20) |
| Mean I/sigma(I) | 8.71 | (2.06) |
| Wilson B-factor (Å2) | 86.06 | |
| R-merge | 0.157 | (0.9816) |
| R-meas | 0.1737 | (1.082) |
| R-pim | 0.07385 | (0.4537) |
| CC1/2 | 0.994 | (0.683) |
| CC* | 0.998 | (0.901) |
| Reflections used in refinement | 15628 | (2596) |
| Reflections used for R-free | 786 | (131) |
| R-work | 0.1973 | (0.2797) |
| R-free | 0.2456 | (0.3083) |

| | |
|---|---|
| Number of non-hydrogen atoms | 4883 |
| macromolecules | 4883 |
| ligands | 0 |
| solvent | 0 |
| Protein residues | 637 |
| RMS(bonds) (Å) | 0.001 |
| RMS(angles) (deg) | 0.41 |
| Ramachandran favored (%) | 95.71 |
| Ramachandran allowed (%) | 3.97 |
| Ramachandran outliers (%) | 0.32 |
| Rotamer outliers (%) | 0.18 |
| Clashscore | 3.75 |
| Average B-factor (Å2) | 91.37 |
| macromolecules | 91.37 |

*Statistics for the highest-resolution shell are shown in parentheses.

### Determination of Paratope/Epitope

The paratope: epitope interface was defined using a 5 Å Euclidian distance threshold cutoff (Madsen et al., Comp Struct Biotechnol J 23, 199-211, 2024) for protein contact calculation using the MOE software (CCG) and resulting in identification of the epitope as discontinuous and comprised by amino acids .L26, C30, L32, C33, D34, S35, S36, C40, Q41, T42, E43, G44, W47, S62, C63, V64, S65, L66, P67, E68, N70, A71, F74, F88, T89.

The corresponding paratope comprised heavy chain amino acid residues S28, T30, S31, G32, Y34, Y51, F54, D55, R57, N59, Y101, G102, S103, E104 (continuous numbers) and light chain amino acid residues N31, R33, T34, K36, Y38, W56, S97, Y98, N99, L100, Y102 (continuous numbers).

With reference to the CDRs previously described all CDRs are within a 5 Å interaction distance of one or more paratope residue as illustrated in table 7.2.

TABLE 7.2
CDRs of mAb A with paratope
residues underlined.

| | |
|---|---|
| CDRH1 | SGSYWN (SEQ ID NO.: 62) |
| CDRH2 | YISFDGRNNYNPSLKN (SEQ ID NO.: 64) |
| CDRH3 | DYYGSEGFGY (SEQ ID NO.: 71) |
| CDRL1 | KSSQSLLNSRTRKNYLA (SEQ ID NO.: 73) |
| CDRL2 | WTSTRES (SEQ ID NO.: 76) |
| CDRL3 | KQSYNLPYT (SEQ ID NO.: 78) |

### Example 7b: Epitope Mapping of V12, the Anti-ALK7 Fab of V12, Using X-Ray Crystallography.

### Crystallisation

The Fab fragment of V12 was prepared as described above and was mixed in a 1:2 molar ratio with ALK7 ECD. The complex was subjected to size exclusion chromatography on a HiLoad 16/60 Superdex 75 μg column (GE Healthcare) run with 20 mM HEPES, pH 7.5, 140 mM NaCl buffer. The fractions containing the Fab/ALK7 ECD complex were pooled and concentrated to 10.6 mg/ml. Crystals of the Fab/ALK7 ECD complex were grown using sitting drop vapour diffusion at 20° C. The crystal used was grown using a protein solution of 300 nL 8.0 mg/mL complex in 20 mM HEPES, pH 7.4, 140 mM NaCl mixed with 300 nL of 0.2 M sodium malonate, pH 6.0, 20% (w/v) PEG 3350 as precipitant and incubated over 80 μL precipitant.

### Diffraction Data Collection

The crystal was cryo protected in precipitant added 20% of ethylene glycol prior to flash cooling in liquid nitrogen. Diffraction data were collected at 100K at the European Synchrotron Radiation Facility beamline ID23-1(0.8856 Å wavelength) using an Eiger 16M pixel detector from Dectris. Autoindexing, integration and scaling of the data were performed with programmes from the XDS package (diffracting data statistics are summarised in Table 7.3).

### Crystal Structure Determination and Refinement

The asymmetric unit contains two V12 Fab/ALK7 ECD complexes. The structure was determined by molecular replacement using Phaser as implemented in the programme suite Phenix using a model of V12 Fab created by AlphaFold2 and ALK7 ECD from a previously determined crystal structure as search models. The full structure model was refined using steps of Phenix refinement and manual rebuilding in COOT. The refinement statistics are found in Table 7.3.

TABLE 7.3
Data collection and refinement statistics for the
Fab of V12/ALK7 ECD complex crystal structure.

| Wavelength (Å) | 0.8856 | |
|---|---|---|
| Resolution range (Å) | 47.43-2.11 | (2.16-2.11)* |
| Space group | C2 | |
| Unit cell (Å, deg) | 189.0 110.9 63.9 90 104.5 90 | |
| Total reflections | 552676 | (33577) |
| Unique reflections | 207358 | (12718) |
| Multiplicity | 2.7 | (2.6) |
| Completeness (%) | 99.88 | (99.98) |
| Mean I/sigma(I) | 3.49 | (0.26) |
| Wilson B-factor (Å2) | 38.08 | |
| R-merge | 0.1409 | (2.959) |
| R-meas | 0.1768 | (3.662) |
| R-pim | 0.1056 | (2.134) |
| CC1/2 | 0.987 | (0.0722) |
| CC* | 0.997 | (0.367) |
| Reflections used in refinement | 73211 | (5191) |
| Reflections used for R-free | 1999 | (142) |
| R-work | 0.1793 | (0.2709) |
| R-free | 0.2133 | (0.3059) |

| | |
|---|---|
| Number of non-hydrogen atoms | 8511 |
| macromolecules | 7903 |
| ligands | 28 |
| solvent | 580 |
| Protein residues | 1029 |
| RMS(bonds) (Å) | 0.003 |
| RMS(angles) (deg) | 0.71 |
| Ramachandran favored (%) | 95.76 |
| Ramachandran allowed (%) | 3.94 |
| Ramachandran outliers (%) | 0.30 |
| Rotamer outliers (%) | 0.22 |
| Clashscore | 2.76 |
| Average B-factor (Å2) | 44.64 |
| macromolecules | 44.44 |
| ligands | 72.82 |
| solvent | 45.98 |

*Statistics for the highest-resolution shell are shown in parentheses.

### Determination of Paratope/Epitope

The paratope: epitope interface was defined using the same criteria and method as mentioned above resulting in identification of the epitope as discontinuous and comprised by amino acids L26, C30, L32, C33, S35, S36, C40, Q41, T42, E43, G44, A45, W47, S62, C63, V64, S65, L66, P67, E68, N70, A71, V73, F74, and F88.

The corresponding paratope comprised heavy chain amino acid residues T30, S31, Y34, Y51, F54, D55, R57, N59, Y101, G102, S103, and E104 (continuous numbers) and light chain amino acid residues N31, R33, T34, K36, Y38, W56, S97, Y98, N99, L100, and Y102 (continuous numbers)

With reference to the CDRs previously described all CDRs are within a 5 Å interaction distance of one or more residue as illustrated in Table 7.4

TABLE 7.3
CDRs of mAb V12 with paratope
residues underlined.

| | |
|---|---|
| CDRH1 | SGSYWN (SEQ ID NO.: 62) |
| CDRH2 | YISFDGRTNYNPSLKS (SEQ ID NO.: 66) |
| CDRH3 | DYYGSEGFAY (SEQ ID NO.: 72) |
| CDRL1 | KSSQSLLNSRTRKNYLA (SEQ ID NO.: 73) |
| CDRL2 | WASTRES (SEQ ID NO.: 77) |
| CDRL3 | KQSYNLPYT (SEQ ID NO.: 78) |

To further compare the epitope: paratope interactions, the information is collected in Table 7.5 below.

TABLE 7.5
The epitope and paratope as determined by a distance
of 5 Å or less for the two mAbs.

| mAb | A | V12 |
|---|---|---|
| AA residues | L26, C30, L32, C33, S35, | L26, C30, L32, C33, D34, |
| | S36C40, | S35, S36 |
| | Q41, T42, E43, G44, (A45), | C40, Q41, T42, E43, G44, |
| | W47 | W47 |
| | S62, C63, V64, S65, L66, | S62, C63, V64, S65, L66, |
| | P67, E68, | P67, E68, |
| | L69, N70, A71, (V73), F74 | N70, A71, F74, |
| | F88 | F88, T89 |
| Paratope | T30, S31, Y34, | S28, T30, S31, G32, Y34, |
| residues | Y51, F54, D55, R57, N59 | Y51, F54, D55, R57, N59, |
| Heavy chain | Y101, G102, S103, E104 | Y101, G102, S103, E104 |
| Paratope | N31, R33, T34, K36, Y38, | N31, R33, T34, K36, Y38, |
| residues | W56, | W56, |
| Light chain | S97, Y98, N99, L100, Y102 | S97, Y98, N99, L100, |
| | | Y102 |

( ) indicates residue which differs between entities in same crystal and _ indicates residue(s) which differ between the two mAbs.

The epitopes and paratope as determined for the two mAb variants are almost identical except for a few amino acid residues. The epitope on ALK7-ECD in close contact with the binding region extends from amino acid residue 26 to amino acid residue 88 (for mAb 1) or 89 (for mAb V12) but appears as a discontinuous epitope with no interactions between the antibody and antigen within the area from amino acid residue 48 to 61. At least two regions of interaction extending over amino acid residue 26 to 47 and amino residue 62 to 88 (for mAb 1) or 89 (for mAb V12) are seen. It is further observed that the CDR1 is reaching to the ultima AA's of the epitope i.e. AA 88 (and 89). The 3D epitope thus includes three stretches of the antigen namely aa 26-47, aa 62-74 and aa 88/89 of ALK7.

The CDR L1 contributes to a bent shape of the epitope and allows the antibodies to interface with a turn in the ALK7, while the unique embedding of CDR H2 makes an interaction (W56) with a groove in the ALK7 epitope.

The ECD sequence of ALK7 with the epitope stretches underlined and key epitope residues in bold, as determined by X-ray crystallography.

20 30 40 50 60 70 80
.ELSPGLKCVCLLCDSSNFTCQTEGACWASVMLINGKEQVIKSCVSLPELNAQVFCHSSNN
90 100 110
VTKTECCFTDFCNNITLHLPTASPNAPKLGPME

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

1. An anti-ALK7 antibody or antigen binding fragment thereof comprising:
(a) a heavy chain variable region comprising a hCDR1 of SEQ ID NO.: 62 or SEQ ID NO.: 63 with up to 1 amino acid substitution, a hCDR2 of any one of SEQ ID NOs.: 64-70 with up to 1 or 2 amino acid substitutions, and a hCDR3 of SEQ ID NO.: 71 or SEQ ID NO.: 72 with up to 1 or 2 amino acid substitutions, and
(b) a light chain variable region comprising a ICDR1 of any one of SEQ ID NOs.: 73-75 with up to 1 or 2 amino acid substitutions, ICDR2 of SEQ ID NO.: 76 or SEQ ID NO.: 77 with up to 1 amino acid substitutions, and ICDR3 of SEQ ID NO.: 78 or SEQ ID NO.: 79 with up to 1 amino acid substitution.

2. The anti-ALK7 antibody or antigen binding fragment thereof of claim 1 specifically binding to the extracellular domain of human ALK7, wherein the extracellular domain of human ALK7 comprises amino acid residues 26-113 of human ALK7 of SEQ ID NO.: 82.

3. The anti-ALK7 antibody or antigen binding fragment thereof according to claim 1, comprising:
(a) a heavy chain variable region comprising a hCDR1 of SEQ ID NO.: 62 or SEQ ID NO.: 63, a hCDR2 of any one of SEQ ID NOs.: 64-70, and a hCDR3 of SEQ ID NO.: 71, or SEQ ID NO.: 72, and
(b) a light chain variable region comprising a ICDR1 of any one of SEQ ID NOs.: 73-75, ICDR2 of SEQ ID NO.: 76 or SEQ ID NO.: 77, and ICDR3 of SEQ ID NO.: 78 or SEQ ID NO.: 79.

4. The anti-ALK7 antibody or antigen binding fragment thereof according to claim 1, comprising:
(a) a heavy chain variable region comprising an amino acid sequence having a sequence identity of at least 90% to any one of SEQ ID NOs.: 1-34, and
(b) a light chain variable region comprising an amino acid sequence having a sequence identity of at least 90% to any one of SEQ ID NOs.: 35-59.

5. The anti-ALK7 antibody or antigen binding fragment thereof according to claim 4, comprising:
(a) a heavy chain variable region comprising an amino acid sequence of any one of SEQ ID NOs.: 1-34, and
(b) a light chain variable region comprising an amino acid sequence of any one of SEQ ID NOs.: 35-59.

6. The anti-ALK7 antibody or antigen binding fragment thereof according to claim 3, wherein the anti-ALK7 antibody or antigen binding fragment thereof comprises:
(a) a heavy chain variable region comprising a hCDR1 of SEQ ID NO.: 63, a hCDR2 of SEQ ID NO.: 65, and a hCDR3 of SEQ ID NO.: 72, and
(b) a light chain variable region comprising a ICDR1 of SEQ ID NO.: 74, ICDR2 of SEQ ID NO.: 76, and ICDR3 of SEQ ID NO.: 78.

7. The anti-ALK7 antibody or antigen binding fragment thereof according to claim 1, comprising:
(a) a heavy chain variable region comprising an amino acid sequence of SEQ ID NO.: 18, and
(b) a light chain variable region comprising an amino acid sequence of SEQ ID NO.: 53.

8. An anti-ALK7 antibody or antigen binding fragment thereof according to claim 7, comprising:
(a) a heavy chain variable region comprising an amino acid sequence of SEQ ID NO.: 18,
(b) a light chain variable region comprising an amino acid sequence of SEQ ID NO.: 53,
(c) a heavy chain constant region comprising an amino acid sequence having a sequence identity of at least 98% to any one of SEQ ID NO.: 60, and
(d) a light chain constant region comprising an amino acid sequence having at least 98% sequence identity to SEQ ID NO.: 61.

9. The anti-ALK7 antibody or antigen binding fragment thereof according to claim 1, wherein anti-ALK7 antibody or antigen binding fragment thereof is an IgG1 or IgG4 isotype.

10. An anti-ALK7 antibody or antigen binding fragment thereof binding the extracellular domain of human ALK7 wherein the anti-ALK7 antibody or antigen binding fragment thereof specifically binds a non-contiguous ALK7 epitope that comprises amino acid residues 26-47 and 62-74 of human ALK7 of SEQ ID NO.: 82.

11. The anti-ALK7 antibody or antigen binding fragment thereof according to claim 10, wherein the anti-ALK7 antibody or antigen binding fragment thereof specifically binds a non-contiguous ALK7 epitope that comprises amino acid residues 26-47, 62-74, and 88-89 of human ALK7 of SEQ ID NO.: 82.

12. The anti-ALK7 antibody or antigen binding fragment thereof according to claim 10, wherein the anti-ALK7 antibody or antigen binding fragment thereof binds a non-contiguous ALK7 epitope which does not include amino acid residues 48-61 of human ALK7 of SEQ ID NO.: 82.

13. The anti-ALK7 antibody or antigen binding fragment thereof, binding an ALK7 epitope that comprises at least eight amino acid residues selected from the group consisting of L26, C30, L32, C33, S35, S36, C40, Q41, T42, E43, G44, W47, S62, C63, V64, S65, L66, P67, E68, N70, A71, F74, and F88 relative to human ALK7 of SEQ ID NO: 82.

14. The anti-ALK7 antibody or antigen binding fragment thereof according to claim 13, binding an ALK7 epitope comprising:
(i) optionally the amino acid residue L26 of human ALK7 of SEQ ID NO.: 82,
(ii) at least two amino acid residues selected from the group consisting of C30, L32, C33, S35, S36, C40, Q41, T42, E43, G44, and W47 of human ALK7 of SEQ ID NO.: 82,
(iii) at least two amino acid residues selected from the group consisting of S62, C63, V64, S65, L66, P67, E68, N70, A71, and F74 of human ALK7 of SEQ ID NO.: 82, and
(iv) optionally at least one amino acid residue selected from the group consisting of F88 and T89 of human ALK7 of SEQ ID NO.: 82.

15. The anti-ALK7 antibody or antigen binding fragment thereof according to claim 1, wherein the anti-ALK7 antibody or antigen binding fragment thereof has an IC50 below 50 nM.

16. The anti-ALK7 antibody or antigen binding fragment thereof according to claim 1, wherein the anti-ALK7 antibody or antigen binding fragment thereof has an IC50 below 10 nM.

17. A pharmaceutical composition comprising an anti-ALK7 antibody or antigen binding fragment thereof according to claim 1.

18. A pharmaceutical composition comprising an anti-ALK7 antibody or antigen binding fragment thereof according to claim 6.

19. A method of treating a disease or condition where inhibition of ALK7 signalling is beneficial, comprising administering an anti-ALK7 antibody or antigen binding fragment thereof according to claim 1 to a subject in need thereof, wherein said disease or condition is selected from the group consisting of type 2 diabetes, overweight, obesity and cardiovascular disease (CVD).

20. A method of treating a disease or condition where inhibition of ALK7 signalling is beneficial, comprising administering an anti-ALK7 antibody or antigen binding fragment thereof according to claim 6 to a subject in need thereof, wherein said disease or condition is selected from the group consisting of type 2 diabetes, overweight, obesity and cardiovascular disease (CVD).


SEQUENCE LISTING (88 sequences)

SEQ ID NO: 1 - protein, 119 aa, synthetic construct
DVHLQESGPGLVKPSQSLSLTCSVTGHSITSGNYWNWIRQFPGNKLEWMGYISFDGRNNYNPSLKNRISITRDTSKNQFFLRLNSVTTEDTATYFCARDYYGSEGFGYWGQGTLVTVSA

SEQ ID NO: 2 - protein, 119 aa, synthetic construct
DVQVQESGPGPVKPSQSLSLTCSVTGYSITSGSYWNWIRQFPGNKLEWMGYISFDGRNNYNPSLKNRISITRDTSKNQFFLKLNSVTTEDTATYYCARDYYGSEGFAYWGQGTLVTVSA

SEQ ID NO: 3 - protein, 119 aa, synthetic construct
  VARIANT 1: note = Glutamine or pyroglutamic acid
XVHLQESGPGLVKPSQSLSLTCSVSGSSITSGNYWNWIRQFPGNGLEWMGYISFDGRTNYNPSLKNRISITRDTSKNQFSLRLNSVTTEDTATYFCARDYYGSEGFGYWGQGTLVTVSA

SEQ ID NO: 4 - protein, 119 aa, synthetic construct
  VARIANT 1: note = Glutamine or pyroglutamic acid
XVQLQESGPGDVKPSQSLSLTCSVEGYSITSGSYWNWIRQFPGNGLEWIGYISFDGRTNYNPSLKNRISITRDTSKNQFFLKLNSVTTYDTATYYCARDYYGSEGFAYWGQGTLVTVSA

SEQ ID NO: 5 - protein, 119 aa, synthetic construct
  VARIANT 1: note = Glutamine or pyroglutamic acid
XVQVQESGPGPVKPSQSLSLTCSVTGYSITSGSYWNWIRQFPGNGLEWMGYISFDGRTNYNPSLKSRISITRDTSKNQFFLKLNSVTTEDTATYYCARDYYGSEGFAYWGQGTLVTVSA

SEQ ID NO: 6 - protein, 119 aa, synthetic construct
  VARIANT 1: note = Glutamine or pyroglutamic acid
XVQLQESGPGLVKPSQTLSLTCAVPGGSFSSGSYWNWIRQPPGKGLEWIGYISFDGRRNYNPSLKNRVTISVDTSKNQFSLKLSSVTAADTAVYYCARDYYGSEGFAYWGQGTLVTVSS

SEQ ID NO: 7 - protein, 119 aa, synthetic construct
  VARIANT 1: note = Glutamine or pyroglutamic acid
XVQLQESGPGLVKPSQTLSLTCAVSGASITSGNYWNWIRQPPGKGLEWIGYISFDGRTNYNPSLKNRVSISVDTSKNQFSLRLNSVTAADTAVYFCARDYYGSEGFGYWGQGTLVTVSS

SEQ ID NO: 8 - protein, 119 aa, synthetic construct
  VARIANT 1: note = Glutamine or pyroglutamic acid
XVQLQESGPGLVKPSETLSLTCTVSGYSITSGSYWNWIRQPPGKGLEWIGYISFDGRNNYNPSLKNRVTISVDTSKNQFSLKLSSVTAADTAVYYCARDYYGSEGFAYWGQGTLVTVSS

SEQ ID NO: 9 - protein, 119 aa, synthetic construct
EVQLVESGGGLVQPGGSLRLSCAASGYSITSGSYWNWIRQAPGKGLEWMGYISFDGRNNYNPSLKNRITISRDTSKNTFYLQMNSLRAEDTAVYYCARDYYGSEGFAYWGQGTLVTVSS

SEQ ID NO: 10 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSA

SEQ ID NO: 11 - protein, 119 aa, synthetic construct
  VARIANT 1: note = Glutamine or pyroglutamic acid
XVQLQESGPGLVKPSQSLSLTCSVEGYSITSGSYWNWIRQFPGKGLEWIGYISFDGRTNYNPSLKNRITITRDTSKNQFSLKLNSVTAADTAVYYCARDYYGSEGFGYWGQGTLVTVSA

SEQ ID NO: 12 - protein, 119 aa, synthetic construct
  VARIANT 1: note = Glutamine or pyroglutamic acid
XVQLQESGPGLVKPSQSLSLTCSVSGYSITSGSYWNWIRQPPGKGLEWMGYISFDGRTNYNPSLKSRISISVDTSKNQFFLKLNSVTTEDTAVYYCARDYYGSEGFAYWGQGTLVTVSA

SEQ ID NO: 13 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 14 - protein, 119 aa, synthetic construct
  VARIANT 1: note = Glutamine or pyroglutamic acid
XVQLQESGPGDVKPSQSLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 15 - protein, 119 aa, synthetic construct
  VARIANT 1: note = Glutamine or pyroglutamic acid
XVQLQESGPGDVKPSQSLSLTCSVEGYSITSGSYWNWIRQPPGKGLEWIGYISFDGRTNYNPSLKNRISISRDTSKNQFSLKLNSVTAADTATYYCARDYYGSEGFAYWGQGTLVTVSS

SEQ ID NO: 16 - protein, 119 aa, synthetic construct
  VARIANT 1: note = Glutamine or pyroglutamic acid
XVQLQESGPGLVKPSQSLSLTCSVSGYSITSGSYWNWIRQPPGKGLEWMGYISFDGRTNYNPSLKSRISISRDTSKNQFFLKLNSVTTEDTAVYYCARDYYGSEGFAYWGQGTLVTVSA

SEQ ID NO: 17 - protein, 119 aa, synthetic construct
  VARIANT 1: note = Glutamine or pyroglutamic acid
XVQLQESGPGLVKPSQSLSLTCSVSGYSITSGSYWNWIRQPPGKGLEWIGYISFDGRTNYNPSLKSRISISRDTSKNQFFLKLSSVTTEDTAVYYCARDYYGSEGFAYWGQGTLVTVSS

SEQ ID NO: 18 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISRDTSKNQFSLKLSSVTAADTAVYYCARDYYGSEGFAYWGQGTLVTVSS

SEQ ID NO: 19 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISVDTSKNQFSLKLSSVTAADTATYYCARDYYGSEGFAYWGQGTLVTVSS

SEQ ID NO: 20 - protein, 119 aa, synthetic construct
  VARIANT 1: note = Glutamine or pyroglutamic acid
XVQLQESGPGLVKPSETLSLTCTVSGYSITSGSYWNWIRQPPGKGLEWIGYISFSGRNNYNPSLKNRVTISVDTSKNQFSLKLSSVTAADTAVYYCARDYYGSEGFAYWGQGTLVTVSS

SEQ ID NO: 21 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFDGRTNYNPSLKNRVTISRDTSKNQFSLKLNSVTAADTAVYYCARDYYGSEGFAYWGQGTLVTVSS

SEQ ID NO: 22 - protein, 119 aa, synthetic construct
  VARIANT 1: note = Glutamine or pyroglutamic acid
XVQLQESGPGLVKPSQTLSLTCSVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 23 - protein, 119 aa, synthetic construct
  VARIANT 1: note = Glutamine or pyroglutamic acid
XVQLQESGPGLVKPSQSLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 24 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFDGRTNYNPSLKNRVTISRDTSKNQFSLKLSSVTAADTAVYYCARDYYGSEGFAYWGQGTLVTVSS

SEQ ID NO: 25 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISRDTSKNQFSLKLNSVTAADTAVYYCARDYYGSEGFAYWGQGTLVTVSS

SEQ ID NO: 26 - protein, 119 aa, synthetic construct
  VARIANT 1: note = Glutamine or pyroglutamic acid
XVQLQESGPGLVKPSETLSLTCTVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 27 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 28 - protein, 119 aa, synthetic construct
  VARIANT 1: note = Glutamine or pyroglutamic acid
XVQLQESGPGLVKPSETLSLTCSVEGYSITSGSYWNWIRQPPGKGLEWIGYISFTGRTNYNPSLKNRVTISRDTSKNQFSLKLNSVTAADTATYYCARDYYGSEGFAYWGQGTLVTVSS

SEQ ID NO: 29 - protein, 119 aa, synthetic construct
  VARIANT 1: note = Glutamine or pyroglutamic acid
XVQLQESGPGLVKPSQSLSLTCSVEGYSITSGSYWNWIRQPPGKGLEWIGYISFSGRTNYNPSLKNRVTISRDTSKNQFSLKLNSVTAADTATYYCARDYYGSEGFAYWGQGTLVTVSS

SEQ ID NO: 30 - protein, 119 aa, synthetic construct
  VARIANT 1: note = Glutamine or pyroglutamic acid
XVQLQESGPGLVKPSETLSLTCSVEGYSITSGSYWNWIRQPPGKGLEWIGYISFSGRTNYNPSLKNRVTISRDTSKNQFSLKLNSVTAADTATYYCARDYYGSEGFAYWGQGTLVTVSS

SEQ ID NO: 31 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISVDTSKNQFSLKLSSVTAADTAVYYCARDYYGSEGFAYWGQGTLVTVSS

SEQ ID NO: 32 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 33 - protein, 119 aa, synthetic construct
  VARIANT 1: note = Glutamine or pyroglutamic acid
XVQLQESGPGLVKPSESLSLTCTVEGYSITSGSYWNWIRQPPGKGLEWIGYISFDGRTNYNPSLKNRVTISRDTSKNQFSLKLSSVTAADTATYYCARDYYGSEGFAYWGQGTLVTVSS

SEQ ID NO: 34 - protein, 119 aa, synthetic construct
  VARIANT 1: note = Glutamine or pyroglutamic acid
XVQLQESGPGLVKPSQTLSLTCTVEGYSITSGSYWNWIRQPPGKGLEWIGYISFDGRTNYNPSLKNRVTISRDTSKNQFSLKLNSVTAADTATYYCARDYYGSEGFAYWGQGTLVTVSS

SEQ ID NO: 35 - protein, 113 aa, synthetic construct
DIVMSQSPSSLAVSAGEKVTMSCKSSQSLLNSRTRKNYLAWFQQKPGQSPKLLIYWTSTRESGVPDRFTGSGSGTDFTLTISSVQAEDLAVYYCKQSYNLPYTFGGGTKLEIK

SEQ ID NO: 36 - protein, 113 aa, synthetic construct
DNVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPYTFGGGTKLEIK

SEQ ID NO: 37 - protein, 113 aa, synthetic construct
DIVMSQSPSSLAVSAGEKVTMSCKSSQSLLNSRTRKNYLAWFQQKPGQSPKLLIYWTSTRESGVPDRFTGSGSGTDFTLTISSVQAEDLAVYYCKRSYNLPYTFGGGTKLEIK

SEQ ID NO: 38 - protein, 113 aa, synthetic construct
DIVMTQSPMSLAVSLGERATINCKSSQSLLVSRTRKNYLAWYQQKPGQPPKLLIYWASTRESGVPDRFSGSGSGTDFTLTISSLQAEDVAVYYCKQSYNLPYTFGQGTKLEIK

SEQ ID NO: 39 - protein, 113 aa, synthetic construct
DIVMTQSPISLAVSLGERATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 40 - protein, 113 aa, synthetic construct
DIVMTQSPDSLAVSAGEHVTINCKSSQSLLVSRTRKNYLAWYQQKPGQPPKLLIYWASTRESGVPDRFSGSGSGTDFTLTISSVQAEDVAVYYCKQSYNLPYTFGQGTKLEIK

SEQ ID NO: 41 - protein, 113 aa, synthetic construct
DIVMSQSPSSLAVSLGEDVTMHCKSSQSLLNSRTRKNYLAWYQQKPGQSPKLLIYWASTRESGVPDRFTGSGSGTDFTLTISSVQAEDLAVYYCKQSYNLPYTFGEGTKLEIK

SEQ ID NO: 42 - protein, 113 aa, synthetic construct
DIVMTQSPYSLAVSLGERATINCKSSQSLLYSRTRKNYLAWYQQKPGQPPKLLIYWASTRESGVPDRFSGSGSGTDFTLTISSLQAEDVAVYYCKQSYNLPYTFGQGTKLEIK

SEQ ID NO: 43 - protein, 113 aa, synthetic construct
DIVMTQSPDSLAVSLGERATINCKSSQSLLNSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 44 - protein, 113 aa, synthetic construct
DIVMTQSPDSLAVSLGERATINCKSSQSLLNSRTRKNYLAWYQQKPGQPPKLLIYWTSTRESGVPDRFSGSGSGTDFTLTISSLQAEDVAVYYCKQSYNLPYTFGGGTKVEIK

SEQ ID NO: 45 - protein, 113 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLPYTFGGGTKVEIK

SEQ ID NO: 46 - protein, 113 aa, synthetic construct
DIVMSQSPDSLAVSLGEDVTMHCKSSQSLLNSRTRKNYLAWYQQKPGQSPKLLIYWASTRESGVPDRFTGSGSGTDFTLTISSVQAEDVAVYYCKQSYNLPYTFGQGTKLEIK

SEQ ID NO: 47 - protein, 113 aa, synthetic construct
DIVMTQSPISLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 48 - protein, 113 aa, synthetic construct
DIVMTQSPISLAVSLGERATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 49 - protein, 113 aa, synthetic construct
DIVMTQSPDSLAVSLGERVTINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 50 - protein, 113 aa, synthetic construct
DIVMTQSPDSLAVSLGERATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 51 - protein, 113 aa, synthetic construct
DIVMTQSPDSLAVSLGEDVTMHCKSSQSLLYSRTRKNYLAWYQQKPGQPPKLLIYWASTRESGVPDRFTGSGSGTDFTLTISSVQAEDVAVYYCKQSYNLPYTFGQGTKLEIK

SEQ ID NO: 52 - protein, 113 aa, synthetic construct
DIVMTQSPDSLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 53 - protein, 113 aa, synthetic construct
DIVMTQSPDSLAVSLGEDATINCKSSQSLLVSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 54 - protein, 113 aa, synthetic construct
DIVMTQSPDSLAVSLGERATINCKSSQSLLVSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 55 - protein, 113 aa, synthetic construct
DIVMTQSPDSLAVSLGEDATINCKSSQSLLYSRTRKNYLAWFQQKPGQSPKLLIYWTSTRESGVPDRFSGSGSGTDFTLTISSLQAEDVAVYYCKQSYNLPYTFGGGTKVEIK

SEQ ID NO: 56 - protein, 113 aa, synthetic construct
DIVMTQSPDSLAVSLGEDATINCKSSQSLLYSRTRKNYLAWFQQKPGQPPKLLIYWTSTRESGVPDRFSGSGSGTDFTLTISSLQAEDVAVYYCKQSYNLPYTFGGGTKVEIK

SEQ ID NO: 57 - protein, 113 aa, synthetic construct
DIVMTQSPDSLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGQGTKLEIK

SEQ ID NO: 58 - protein, 113 aa, synthetic construct
DIVMTQSPDSLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 59 - protein, 113 aa, synthetic construct
DIVMTQSPISLAVSLGEDATINCKSSQSLLYSRTRKNYLAWYQQKPGQPPKLLIYWTSTRESGVPDRFSGSGSGTDFTLTISSLQAEDVAVYYCKQSYNLPYTFGGGTKLEIK

SEQ ID NO: 60 - protein, 330 aa, synthetic construct
  VARIANT 330: note = May be absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 107 aa, synthetic construct [sequence omitted]

SEQ ID NO: 62 - protein, 6 aa, synthetic construct
SGNYWN

SEQ ID NO: 63 - protein, 6 aa, synthetic construct
SGSYWN

SEQ ID NO: 64 - protein, 16 aa, synthetic construct
YISFDGRNNYNPSLKN

SEQ ID NO: 65 - protein, 16 aa, synthetic construct
YISFDGRTNYNPSLKN

SEQ ID NO: 66 - protein, 16 aa, synthetic construct
YISFDGRTNYNPSLKS

SEQ ID NO: 67 - protein, 16 aa, synthetic construct
YISFDGRRNYNPSLKN

SEQ ID NO: 68 - protein, 16 aa, synthetic construct
YISFTGRTNYNPSLKN

SEQ ID NO: 69 - protein, 16 aa, synthetic construct
YISFSGRNNYNPSLKN

SEQ ID NO: 70 - protein, 16 aa, synthetic construct
YISFSGRTNYNPSLKN

SEQ ID NO: 71 - protein, 10 aa, synthetic construct
DYYGSEGFGY

SEQ ID NO: 72 - protein, 10 aa, synthetic construct
DYYGSEGFAY

SEQ ID NO: 73 - protein, 17 aa, synthetic construct
KSSQSLLNSRTRKNYLA

SEQ ID NO: 74 - protein, 17 aa, synthetic construct
KSSQSLLVSRTRKNYLA

SEQ ID NO: 75 - protein, 17 aa, synthetic construct
KSSQSLLYSRTRKNYLA

SEQ ID NO: 76 - protein, 7 aa, synthetic construct
WTSTRES

SEQ ID NO: 77 - protein, 7 aa, synthetic construct
WASTRES

SEQ ID NO: 78 - protein, 9 aa, synthetic construct
KQSYNLPYT

SEQ ID NO: 79 - protein, 9 aa, synthetic construct
KRSYNLPYT

SEQ ID NO: 80 - protein, 108 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 81 - protein, 107 aa, synthetic construct [sequence omitted]

SEQ ID NO: 82 - protein, 493 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 83 - protein, 99 aa, synthetic construct
LKCVCLLCDSSNFTCQTEGACWASVMLTNGKEQVIKSCVSLPELNAQVFCHSSNNVTKTECCFTDFCNNITLHLPTASPNAPKLGPMEGGGHHHHHHHH

SEQ ID NO: 84 - protein, 713 aa, synthetic construct
LKCVCLLCDSSNFTCQTEGACWASVMLTNGKEQVIKSCVSLPELNAQVFCHSSNNVTKTECCFTDFCNNITLHLPTASPNAPKLGPMEGGGGQGGGGQGGGGQDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGQGGGGQGGGGQGGHHHHHHHH

SEQ ID NO: 85 - protein, 331 aa, synthetic construct
LKCVCLLCDSSNFTCQTEGACWASVMLTNGKEQVIKSCVSLPELNAQVFCHSSNNVTKTECCFTDFCNNITLHLPTASPNAPKLGPMEGGGGAEPGAEPGAEPGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPCVKFNWYVDGVEVHNAKTKPCEEQYNSTYRCVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 329 aa, synthetic construct; identical to SEQ ID NO: 86

SEQ ID NO: 88 - protein, 93 aa, Homo sapiens
ELSPGLKCVCLLCDSSNFTCQTEGACWASVMLTNGKEQVIKSCVSLPELNAQVFCHSSNNVTKTECCFTDFCNNITLHLPTASPNAPKLGPME